(19) United States
(12) Patent Application Publication
(10) Pub. No.: US 2025/0163161 A1
(43) Pub. Date: May 22, 2025

(54) "TWO-IN-ONE"-ANTIBODIES BINDING TO EGFR/PD-L1-DOUBLE POSITIVE CELLS

(71) Applicant: TECHNISCHE UNIVERSITÄT DARMSTADT, Darmstadt (DE)

(72) Inventors: Harald Kolmar, Mühltal (DE); Julia Harwardt, Weiterstadt (DE); Jan Patrick Bogen, Darmstadt (DE); Stefania Carrara, Mühltal (DE); Michael Ulitzka, Darmstadt (DE)

(21) Appl. No.: 18/842,770

(22) PCT Filed: Feb. 15, 2023
(86) PCT No.: PCT/EP2023/053749

§ 371 Date: Aug. 29, 2024

(30) Foreign Application Priority Data
Mar. 1, 2022 (EP) 22159491.4
Sep. 20, 2022 (EP) 22196556.9

(51) Int. Cl. C07K 16/28; A61P 29/00

(52) U.S. Cl. CPC C07K 16/2863; A61P 29/00; C07K 16/2827; C07K 2317/14; C07K 2317/24; C07K 2317/31; C07K 2317/55; C07K 2317/565; C07K 2317/76; C07K 2317/92; C07K 2317/94

(57) ABSTRACT

The present invention is related to a at least bispecific monoclonal antibody or antigen-binding fragment, or a substantially similar variant thereof characterized in that at least one Fab-region binds specifically to epidermal growth factor receptor (EGFR) and programmed death-ligand 1 (PD-L1). Multispecific antibodies are also disclosed, making use of the inventive “two-in-one” scaffold for specific applications.

### FIELD OF THE INVENTION

Evolutionary pressure has selected antibodies as key immune molecules acting against foreign pathogens. The development of monoclonal antibody technology has allowed their widespread use in research, real-time diagnosis and treatment of multiple diseases, including cancer.

However, compared with hematologic malignancies, solid tumours have often proven to be relatively resistant to antibody-based therapies. In an attempt to improve the tumour-targeting efficacy of antibodies, new formats with modified, multivalent properties have been generated.

Initially, these formats imitated the structure of native IgG, creating mostly monospecific, bivalent antibodies. Recently, novel multivalent antibodies have been developed to maximize tumour targeting capabilities through enhanced biodistribution and functional affinity.

In an attempt to improve tumour penetration, recombinant antibodies with modified properties have been generated. Novel antibody formats, such as the single-chain Fv (scFv; 25-30 kDa), exhibit better pharmacokinetics than intact IgGs for tissue penetration, and enable binding specificity to be encoded by a single gene.

The covalent conjugation of small recombinant antibody molecules with poly(ethylene glycol) (PEG)—also known as ‘PEGylation’—is a well-established procedure to improve serum half-life by increasing the molecular mass and the hydrodynamic radius. Another approach is the genetic fusion of antibody fragments to albumin or albumin-binding proteins. This strategy is based on the observation that albumin has a half-life similar to that of IgG and also utilizes FcRn-mediated recycling processes.

Alternatively, the use of bispecific antibodies to recruit serum IgG provides the Fc-associated effector functions and prolongs residence time in serum. Multimerization is a particularly appealing approach to optimize antibody size for the desired pharmacokinetics, and to maximize tumour uptake by enhancing the functional affinity (avidity) of antibody fragments.

One class of new antibodies are the so-called: “Two-in-One antibodies”, which are antibodies in which each Fab arm can bind to two different antigens.

In this application we present a new method for generating avian-derived “Two-in-One antibodies” which possess several advantageous properties as well as a bispecific monoclonal antibody or antigen-binding fragment, or a substantially similar variant thereof characterized in that at least one Fab-region binds specifically to epidermal growth factor receptor (EGFR) and programmed death-ligand 1 (PD-L1).

### BACKGROUND OF THE INVENTION

The IgG-like architecture of “Two-in-One antibodies” accounts for low immunogenicity and also circumvents laborious engineering and purification steps to facilitate correct chain pairing. In this application the identification of Two-in-One antibody by yeast surface display (YSD) screening of a chicken-derived immune library is described. The resulting antibody simultaneously targets the epidermal growth factor receptor (EGFR) and programmed death ligand 1 (PD-L1) at the same Fv fragment with two non-overlapping paratopes. The dual action Fabs are capable of inhibiting EGFR signalling by binding to dimerization domain II as well as blocking the PD-1/PD-L1 interaction. Furthermore, the Two-in-One antibody demonstrates specific cellular binding properties on EGFR/PD-L1 double positive tumour cells. The presented strategy obviates the need for any additional CDR engineering as described in the prior art and allows for obtaining bifunctional and bispecific antibodies upon animal immunization. Therefore, the disclosed method paves the way for further development of therapeutic antibodies derived from avian immunization with novel and tailor-made binding properties.

### DETAILED DESCRIPTION

In a first embodiment, the present invention pertains to a bispecific monoclonal antibody or antigen-binding fragment, or a substantially similar variant thereof characterized in that at least one Fab-region binds specifically to epidermal growth factor receptor (EGFR) and programmed death-ligand 1 (PD-L1). In one embodiment the present invention pertains to the antibody “HCP-LCE” (heavy chain PD-L1—light chain EGFR), however, also the “HCE-LCP” (heavy chain EGFR—light chain PD-L1)-variant is encompassed.

In one embodiment the bispecific monoclonal antibody or antigen-binding fragment, or a substantially similar variant thereof is preferred which binds specifically to at least one of the domains of epidermal growth factor receptor (EGFR) and to at least one of the domains of programmed death-ligand 1 (PD-L1).

In this respect it is important to note that the binding is at the same time and within one Fab-region, so that for example the other Fab-region is available for binding to at least one, preferably two or more further targets.

Further advantages of the two-in-one antibodies of the present invention:
i) tetravalent binding in the two-in-one format due to simultaneous binding within a single Fab-region leading to avidity effects, increased binding efficacy and widening of therapeutic window; and/or
ii) binding to EGFR and PD-L1 results in increased specificity to tumor cells and diminishes adverse effects (e.g., skin rash with anti-EGFR monospecific antibodies) by binding to e.g., NHEK cells with a 10-fold decrease in affinity; and/or
iii) iii) ease of production/manufacturing as a generic IgG antibody structure is used for which platform technologies are established, as compared to other bispecific antibodies with KiH mutations requiring two-step purifications.

The term “antibody”, as used herein, is intended to refer any antigen binding molecule, in some embodiments to immunoglobulin molecules comprised of four polypeptide chains, two heavy (H) chains and two light (L) chains interconnected by disulphide bonds. Each heavy chain is comprised of a heavy chain variable region (“HCVR” or “VH”) and a heavy chain constant region (comprised of domains CH1, CH2 and CH3). Each light chain is comprised of a light chain variable region (“LCVR or “VL”) and a light chain constant region (CL). The VH and VL regions can be further subdivided into regions of hypervariability, termed “complementarity determining regions” (CDR), interspersed with regions that are more conserved, termed “framework regions” (FR). Each VH and VL is composed of three CDRs and four FRs, arranged from amino-terminus to carboxy-terminus in the following order: FR1, CDR1, FR2, CDR2, FR3, CDR3, FR4.

Substitution of up to one, up to two or up to three amino acid residues per CDR, of up to two, up to three or up to four amino acid residues within all three CDRs of one light or heavy chain or omission of one or more CDRs is also possible.

In one embodiment up to one, up to two or up to three amino acid residues are substituted in HCDR1.

In another embodiment up to one, up to two or up to three amino acid residues are substituted in H-CDR2. In yet another embodiment up to one, up to two or up to three amino acid residues are substituted in H-CDR3. In one embodiment up to one, up to two or up to three amino acid residues are substituted in L-CDR1. In another embodiment up to one, up to two or up to three amino acid residues are substituted in L-CDR2. In yet another embodiment up to one, up to two or up to three amino acid residues are substituted in L-CDR3.

Antibodies have been described in the scientific literature in which one or two CDRs can be dispensed with for binding.

If a CDR or amino acid residue(s) thereof is omitted, it is usually substituted with an amino acid occupying the corresponding position in another human antibody sequence or a consensus of such sequences.

In one embodiment up to one, up to two or up to three amino acid residues are omitted in HCDR1.

In another embodiment up to one, up to two or up to three amino acid residues are omitted in H-CDR2. In yet another embodiment up to one, up to two or up to three amino acid residues are omitted in H-CDR3. In one embodiment up to one, up to two or up to three amino acid residues are omitted in L-CDR1. In another embodiment up to one, up to two or up to three amino acid residues are omitted in L-CDR2. In yet another embodiment up to one, up to two or up to three amino acid residues are omitted in L-CDR3.

Positions for substitution within CDRs and amino acids to substitute can also be selected empirically.

Empirical substitutions can be conservative or non-conservative substitutions. In some embodiments also in other parts of the antibody or antigen-fragment thereof amino acids may be exchanged by conservative amino acid substitution. Such a sequence variant, which differs from another sequence only by conservative amino acid substitution(s), is called herein “substantially similar variant”.

A “conservative amino acid substitution” is one in which an amino acid residue is substituted by another amino acid residue having a side chain (R group) with similar chemical properties (e.g., similar charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art.

Examples of groups of amino acids that have side chains with similar chemical properties, and whose substitution for each other constitutes conservative substitutions, include 1) aliphatic side chains: glycine, alanine, valine, leucine and isoleucine; 2) aliphatic hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulphur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine.

Alternatively, a conservative replacement is any change having a positive value in the PAM250 log-likelihood matrix disclosed in Gonnet et al. (1992) Science 256: 1443 45. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions.

For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof.

Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra).

Another preferred algorithm when comparing a sequence of the invention to a database containing many sequences from different organisms is the computer program BLAST, especially BLASTP or TBLASTN, using default parameters. See, e.g., Altschul et al. (1990) J. Mol. Biol. 215: 403 410 and (1997) Nucleic Acids Res. 25:3389 402.

The term “sequence identity” when referring to a nucleic acid or antigen-binding fragment thereof, indicates that, when optimally aligned with appropriate nucleotide insertions or deletions with another nucleic acid (or its complementary strand), there is nucleotide sequence identity in at least about 90%, and more preferably at least about 95%, at least about 96%, at least about 97%, at least about 98% or at least about 99% of the nucleotide bases, as measured by any well-known algorithm of sequence identity, such as FASTA, BLAST or GAP, as discussed.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, at least 98% or at least 99% sequence identity. Preferably, residue positions which are not identical differ by conservative amino acid substitutions.

In some embodiments, the invention is a monoclonal antibody, a polyclonal antibody, a recombinant antibody, a human antibody, a humanized antibody, a chimeric antibody, a multispecific antibody, or an antigen-binding fragment thereof. In some embodiments, the isolated antigen binding protein is a Fab fragment, a Fab′ fragment, a F(ab′)2 fragment, a Fv fragment, a diabody, a nanobody or a single chain antibody molecule. In some embodiments, the isolated antigen binding protein is of the IgG1-, IgG2- IgG3- or IgG4-type. In some embodiments the antibody may be of IgA-, IgD-, IgE- or IgM-type.

In one embodiment the invention is a monoclonal antibody of IgG1-Type. In another embodiment the invention is a scFV-fragment.

The term “human antibody”, as used herein, is intended to include antibodies having variable and constant regions derived from human germline immunoglobulin sequences. The human mAbs of the invention may include amino acid residues not encoded by human germline immunoglobulin sequences (e.g., mutations introduced by random or site-specific mutagenesis in vitro or by somatic mutation in vivo), for example in the CDRs.

The term “antibody fragment”, as used herein, refers to one or more fragments of an antibody that retains the ability to specifically bind to S1-protein and/or RBD. An “antibody fragment” may include a Fab fragment, a F(ab′)2 fragment, a Fv fragment (including scFv-fragments), a dAb fragment, a fragment containing a CDR, or an isolated CDR.

The term “specifically binds” or “binds specifically” or the like, means that an antibody or antigen-binding fragment thereof forms a complex with an antigen that is relatively stable under physiologic conditions. Specific binding can be characterized by an medium to high affinity equilibrium dissociation constant “K_{D}”. The term “K_{D}”, as used herein, is intended to refer to the equilibrium dissociation constant of a particular antibody-antigen interaction.

“High affinity” binding can be characterized by an equilibrium dissociation constant of 1×10⁻⁷ M or less, more preferably of 1×10⁻⁸ M or less, even more preferably of 1×10⁻⁹ M or less; most preferably of 1×10⁻¹⁰ M or less (e.g., a smaller KD denotes a tighter binding) as measured by surface plasmon resonance, e.g. BIACORE™, biolayer interferometer (BLI), SPRi, solution-affinity ELISA. The term “medium affinity” can be characterized by an equilibrium dissociation constant of 1×10⁻³ M or less, preferably 1×10⁻⁴ M or less, more preferably of 1×10⁻⁵ M or less, more preferably of 1×10⁻⁶ M or less.

It is one finding of the present invention that antibodies with “medium affinity” for each target are preferred, since they reduce the chance of binding to cells which are not double-positive for both targets (EGFR and PD-L1), but only display one of the targets at the surface. As such side-effects of the inventive antibodies are reduced and the specificity for double-positive tumour cells is increased. Thus, in one embodiment it is preferred, that only by binding to both targets on the cell surface the affinity is high enough to elicit inhibition of EGFR and PD-L1 activity and/or to use the inventive antibodies for identification, prevention and/or treatment of a disease or condition in a patient.

Thus, in another aspect of the present invention the K_{D} for each single binding to each target (EGFR or PD-L1) is 2-fold, preferably 5-fold, preferably 10-fold, preferably 20-fold, preferably 50-fold, preferably 100-fold, preferably 200-fold, preferably 250-fold higher (i.e., shows less affinity) than the combined binding-K_{D} to both targets (EGFR and PD-L1) on one cell.

An isolated antibody or antigen-binding fragment thereof that specifically binds EGFR and/or PD-L1 may, however, still have cross-reactivity to other related antigens, such as the same molecules from other species. Methods for determining whether two molecules specifically bind are well known in the art and include, for example, equilibrium dialysis, surface plasmon resonance, and the like.

In some embodiments of the present invention, the antibodies or antigen-binding fragments of the invention bind preferably medium affinity to each single target (e.g., EGFR and PD-L1), resulting in a combined high affinity to cells displaying both targets.

The term “Bio-Layer Interferometry (BLI)”, as used herein, refers a label-free technology for measuring biomolecular interactions. It is an optical analytical technique that analyses the interference pattern of white light reflected from two surfaces: a layer of immobilized protein on the biosensor tip, and an internal reference layer. Any change in the number of molecules bound to the biosensor tip causes a shift in the interference pattern that can be measured in real-time.

The binding between a ligand immobilized on the biosensor tip surface and an analyte in solution produces an increase in optical thickness at the biosensor tip, which results in a wavelength shift, Δλ, which is a direct measure of the change in thickness of the biological layer. Interactions are measured in real time, providing the ability to monitor binding specificity, rates of association and dissociation, or concentration, with high precision and accuracy.

Only molecules binding specifically to or dissociating from the biosensor can shift the interference pattern and generate a response profile. Unbound molecules, changes in the refractive index of the surrounding medium, or changes in flow rate do not affect the interference pattern.

This is a unique characteristic of bio-layer interferometry and extends its capability to perform in crude samples used in applications for protein-protein interactions, quantitation, affinity, and kinetics.

BLI-systems are commercially available such as for example Octet® QKe (Fortebio/Sartorius GmbH, Göttingen, Germany).

The term “surface plasmon resonance”, as used herein, refers to an optical phenomenon that allows for the analysis of real-time specific interactions by detection of alterations in protein concentrations within a biosensor matrix, for example using the BIACORE™ system.

An “isolated antibody”, as used herein, is intended to refer to an antibody or antigen-binding fragment thereof that is substantially free of other antibody or antigen-binding fragment thereof having different antigenic specificities (e.g., an isolated antibody that specifically binds EGFR and/or PD-L1 is substantially free of mAbs that specifically bind antigens other than EGFR and/or PD-L1).

The term “compete” when used in the context of the antibody or antigen-binding fragment thereof means competition between the reference antibody or antigen-binding fragment thereof in binding specifically to the target protein (also called “antigen”, in the specific case of the invention this is EGFR and/or PD-L1) with other antigen binding molecule(s).

Such antigen-binding molecule may be selected from the list of: natural ligands to EGFR and/or PD-L1; other antibodies (e.g. cetuximab, panitumumab) or antigen-binding fragments thereof; diabodies, nanobodies, anticalins, scab-proteins, single-chain antibody variable regions, single-chain fragments variables (scFv), etc.

This competition may be determined by an assay in which the reference antibody or antigen binding fragment thereof (i.e., one of the antibodies mentioned hereinunder) prevents or inhibits (e.g., reduces) specific binding of EGFR and/or PD-L1 to another antigen-binding molecule, such as for example a ligand, a test-antibody or antigen-binding fragment thereof.

Numerous types of competitive binding assays can be used to determine if one antigen binding protein competes with another, for example:
solid phase direct or indirect radioimmunoassay (RIA), solid phase direct or indirect enzyme immunoassay (EIA), sandwich;
competition assay (see, e.g., Stahli et al., 1983, Methods in Enzymology 9:242-253);
solid phase direct biotin-avidin EIA (see, e.g., Kirkland et al., 1986, J. Immunol. 137:3614-3619)
solid phase direct labelled assay;
solid phase direct labelled sandwich assay (see, e.g., Harlow and Lane, 1988, Antibodies, A Laboratory Manual, Cold Spring Harbor Press);
solid phase direct label RIA using 1-125 label (see, e.g., Morel et al., 1988, Molec. Immunol. 25:7-15);
solid phase direct biotin-avidin EIA (see, e.g., Cheung, et al., 1990, Virology 176:546-552); and direct labelled RIA (Moldenhauer et al., 1990, Scand. J. Immunol. 32:77-82).

Competitive inhibition is measured by determining the amount of label bound to the solid surface or cells in the presence of the test antigen binding protein. Usually, the antigen binding protein is present in excess.

Antigen binding proteins identified by competition assay (competing antigen-binding molecules) include antigen binding proteins binding specifically to the same epitope as the reference antigen binding proteins and antigen binding proteins binding specifically to an adjacent epitope sufficiently proximal to the epitope bound by the reference antigen binding protein for steric hindrance to occur.

Usually, when a competing antigen binding protein is present in excess, it will inhibit (e.g., reduce) specific binding of a reference antigen binding protein to a common antigen by at least 40%, at least 45%, at least 50%, at least 55%, at least 60%, at least 65%, at least 70% or at least 75% or more.

In some instances, binding is inhibited by at least 80%, at least 85%, at least 90%, at least 95%, or at least 97% or more.

In one embodiment binding is inhibited by at least 95%.

In a second embodiment, the present invention pertains to a bispecific monoclonal antibody or antigen-binding fragment or a substantially similar variant thereof, comprising at least one Fv-region binding specifically to epidermal growth factor receptor (EGFR) and programmed death-ligand 1 (PD-L1).

In further embodiments, the present invention pertains to a bispecific monoclonal antibody or antigen-binding fragment or a substantially similar variant thereof, wherein the paratopes, i.e. the CDR-regions binding to EGFR and PD-L1, on said antibody or antigen-binding fragment or a substantially similar variant thereof do not overlap.

The term “does not overlap” or “non-overlapping” refers generally to sequences of the protein which are at least not identical to each other, preferably separated regions of the protein. In case of non-overlapping paratopes it is meant that the sequences on the antibody responsible for binding to one target epitope are different from the sequences on the antibody responsible for binding to a second target epitope. Non-overlapping paratopes have the advantage that the binding of the antibody to one target epitope does not interfere significantly with the binding to a second target epitope. Thus, for example a one-armed antibody, FAB-fragment or Fv-fragment according to the present invention can still bind to at least two targets at the same time, preferably to PD-L1 and EGFR, allowing the combination with further binders to further targets as described hereinunder.

The position of CDRs within a VH or VL region are defined according to Kabat, et al., Sequences of Proteins of Immunological Interest, 5th Ed. Public Health Service, National Institutes of Health, Bethesda, MD (1991). In another embodiment, the method IMGT may be used to define the CDRs (Brochet, X. et al., Nucl. Acids Res. 36, W503-508 (2008). PMID: 18503082. Giudicelli, V., Brochet, X., Lefranc, M.-P., Cold Spring Harb Protoc. 2011 Jun. 1; 2011 (6). pii: pdb.prot5633. doi: 10.1101/pdb.prot5633).

In further embodiments, the present invention pertains to a bispecific monoclonal antibody or antigen-binding fragment or a substantially similar variant thereof, capable of inhibiting EGFR-signalling and blocking the PD-1/PD-L1 interaction.

EGFR (ErbB-1/HER1) belongs to the receptor-family ErbB which comprises four receptors: EGFR (ErbB-1/HER1), ErbB-2 (Neu, HER2), ErbB-3 (HER3), and ErbB-4 (HER4). This receptor tyrosine kinase family (RTK) of proteins has an extracellular ligand-binding domain, hydrophobic transmembrane domain, and a cytoplasmic tyrosine kinase domain. ErbB receptors are activated by growth factors of EGF-family characterized by three disulphide-bonds that confer binding specificity. Additional structural motifs include immunoglobulin-like domains, heparin-binding sites and glycosylation sites. Typical ligands of EGFR are EGF, Transforming growth factor-α (TGF-α), Amphiregulin (AR), Betacellulin (BTC), Heparin-binding epidermal growth factor (HB-EGF) and Epiregulin (EPR).

Receptor activation is triggered by the cascade of events on cell membrane:
1. Ligand binding: Each ligand binds to the extracellular domain of cognate ErbB receptors.
2. Receptor dimerization: Ligand binding induces the formation of receptor homo or hetero dimers.
3. Activation of kinase domain: Autophosphorylation of key tyrosine residues within carboxy terminal tail activates the receptor and acts as docking sites for proteins with Src homology 2 (SH2) and phosphotyrosine binding domain (PTB) to trigger cellular signalling.

The term “specifically blocking and/or inhibiting EGFR and/or EGFR-signalling” as used herein, therefore refers to any inhibition of the above-mentioned signalling cascade, preferably the inhibition of the dimerization of EGFR (step 2).

Programmed cell death protein 1 (PD-1) plays a vital role in inhibiting immune responses and promoting self-tolerance through modulating the activity of T-cells, activating apoptosis of antigen-specific T-cells and inhibiting apoptosis of regulatory T-cells. Programmed cell death-ligand 1 (PD-L1) is a trans-membrane protein that is considered to be a co-inhibitory factor of the immune response, it can combine with PD-1 to reduce the proliferation of PD-1 positive cells, inhibit their cytokine secretion and induce apoptosis. PD-L1 also plays an important role in various malignancies where it can attenuate the host immune response to tumor cells. Based on these perspectives, PD-1/PD-L1 axis is responsible for cancer immune escape and makes a huge effect on cancer therapy.

The term “specifically blocking and/or inhibiting PD-L1 and/or PD-L1-signalling” as used herein, therefore refers to any inhibition of the above-mentioned PD-1/PD-L1 signalling cascade, preferably the inhibition by binding to an at least partially overlapping epitope with durvalumab (MEDI4736).

In further embodiments, the present invention pertains to a bispecific monoclonal antibody or antigen-binding fragment or a substantially similar variant thereof, wherein the EGFR binding is at the dimerization domain II of EGFR and wherein the antibody prevents PD-1 binding and/or competes for binding to PD-L1 with durvalumab.

The term “epitope” is a region of an antigen that is bound by an antibody. Epitopes may be defined as structural or functional. Functional epitopes are generally a subset of the structural epitopes and have those residues that directly contribute to the affinity of the interaction.

Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

In further embodiments, the present invention pertains to a bispecific monoclonal antibody or antigen-binding fragment or a substantially similar variant thereof, in one embodiment at least 90%, preferably at least 95%, most preferably at least 99% sequence identity, comprising
a heavy chain CDR1 (H-CDR1) domain with an amino acid sequence selected from the group consisting of SEQ ID No.: 03; and/or
a heavy chain CDR2 (H-CDR2) domain with an amino acid sequence selected from the group consisting of SEQ ID No.: 04; and/or
a heavy chain CDR3 (H-CDR3) domain with an amino acid sequence selected from the group consisting of SEQ ID No.: 05; and/or
and/or
a light chain CDR1 (L-CDR1) domain with an amino acid sequence selected from the group consisting of SEQ ID No.: 06; and/or
a light chain CDR2 (L-CDR2) domain with an amino acid sequence selected from the group consisting of SEQ ID No.: 07; and/or
a light chain CDR3 (L-CDR3) domain with an amino acid sequence selected from the group consisting of SEQ ID No.: 08.

In yet a further embodiment, the present invention pertains to a bispecific monoclonal antibody or antigen-binding fragment or a substantially similar variant thereof, in one embodiment at least 90%, preferably at least 95%, most preferably at least 99% sequence identity, comprising
a heavy chain CDR1 (H-CDR1) domain with an amino acid sequence selected from the group consisting of SEQ ID No.: 03; and
a heavy chain CDR2 (H-CDR2) domain with an amino acid sequence selected from the group consisting of SEQ ID No.: 04; and
a heavy chain CDR3 (H-CDR3) domain with an amino acid sequence selected from the group consisting of SEQ ID No.: 05; and
and/or
a light chain CDR1 (L-CDR1) domain with an amino acid sequence selected from the group consisting of SEQ ID No.: 06; and
a light chain CDR2 (L-CDR2) domain with an amino acid sequence selected from the group consisting of SEQ ID No.: 07; and
a light chain CDR3 (L-CDR3) domain with an amino acid sequence selected from the group consisting of SEQ ID No.: 08.

In yet a further embodiment, the present invention pertains to a bispecific monoclonal antibody or antigen-binding fragment or a substantially similar variant thereof, in one embodiment at least 90%, preferably at least 95%, most preferably at least 99% sequence identity, comprising
a heavy chain CDR1 (H-CDR1) domain with an amino acid sequence selected from the group consisting of SEQ ID No.: 03; and
a heavy chain CDR2 (H-CDR2) domain with an amino acid sequence selected from the group consisting of SEQ ID No.: 04; and
a heavy chain CDR3 (H-CDR3) domain with an amino acid sequence selected from the group consisting of SEQ ID No.: 05; and
and
a light chain CDR1 (L-CDR1) domain with an amino acid sequence selected from the group consisting of SEQ ID No.: 06; and
a light chain CDR2 (L-CDR2) domain with an amino acid sequence selected from the group consisting of SEQ ID No.: 07; and
a light chain CDR3 (L-CDR3) domain with an amino acid sequence selected from the group consisting of SEQ ID No.: 08.

In further embodiments, the present invention pertains to a bispecific monoclonal antibody or antigen-binding fragment or a substantially similar variant thereof, in one embodiment having at least 90%, preferably at least 95%, most preferably at least 99% sequence identity, comprising
a variable heavy chain VH-region with an amino acid sequence selected from the group consisting of SEQ ID No.: 01;
and/or
a variable light chain VL-region domain with an amino acid sequence selected from the group consisting of SEQ ID No.: 02.

In further embodiments, the present invention pertains to a bispecific monoclonal antibody or antigen-binding fragment or a substantially similar variant thereof, in one embodiment having at least 90%, preferably at least 95%, most preferably at least 99% sequence identity, comprising a variable heavy chain region of the amino acid sequence in SEQ ID No.: 01 and a variable light chain region of the amino acid sequence in SEQ ID No.: 02.

In further embodiments, the present invention pertains to a bispecific monoclonal antibody or antigen-binding fragment or a substantially similar variant thereof, comprising an amino acid sequence at least 95% identical to the amino acid sequence of SEQ ID No.: 01 and/or of SEQ ID No.: 02.

In further embodiments, the present invention pertains to a bispecific monoclonal antibody or antigen-binding fragment or a substantially similar variant thereof, characterized by one or more of the following:
human or humanized, preferably grafted into a human IgG1-scaffold;
also capable of binding to at least a third target protein selected from the group comprising Fcγ-receptor IIIa/IIIb (CD16a/b), cluster of differentiation 155 (CD155), also known as the poliovirus receptor, MHC class I chain-related proteins A and B (MicA/MicB), cluster of differentiation 159 (CD159), a NKG2 receptor for natural killer cells (NK cells) (there are 7 NKG2 types: A, B, C, D, E, F and H; NKG2D is an activating receptor on the NK cell surface, NKG2A dimerizes with CD94 to make an inhibitory receptor), cluster of differentiation 276 (CD276) or B7 homolog 3 (B7-H3), hepatitis A virus cellular receptor 2 (HAVCR2), also known as T-cell immunoglobulin and mucin-domain containing-3 (TIM-3), T-cell immuno-receptor with Ig and ITIM domains (TIGIT), B-cell maturation antigen (BCMA or BCM), also known as tumour necrosis factor receptor superfamily member 17 (TNFRSF17), interleukin-3 receptor (CD123), natural cytotoxicity triggering receptor 3 (NKp30) or (CD337), natural cytotoxicity triggering receptor 3 (NKp44) or (CD336), natural cytotoxicity triggering receptor 3 (NKp46) or (CD335), cluster of differentiation 27 (CD27), cluster of differentiation 96 (CD96) or Tactile (T-cell activation, increased late expression), NKRP1A, cluster of differentiation 355 (CD355) or Cytotoxic and regulatory T-cell molecule (CRTAM), and lymphocyte function-associated antigen 1 (LFA-1), and/or any combination thereof;
capable of binding specifically to PD-L1-protein with “medium affinity” i.e. an affinity (KD) of 500 nM or less, preferably of 350 nM or less, more preferably of 250 nM or less, even more preferably of 100 nM or less; in some embodiments between 0.1 and 500 nM, preferably between 1 and 450 nM, preferably between 5 and 250 nM, more preferably between 10 and 150 nM, even more preferably between 50 and 130 nM; in some embodiments about 78.3 nM, in some embodiments about 79.5 nM, in some embodiments about 83 nM, in some embodiments about 97.6 nM, in some embodiments about 104 nM, in some embodiments about 115 nM, in some embodiments about 125 nM, in some embodiments about 127 nM.
capable of binding specifically to EGFR-protein with “medium affinity” i.e. an affinity (KD) of 1000 nM or less, more preferably 800 nM or less; more preferably 700 nM or less; in some embodiments between 50 and 1000 nM, preferably between 100 and 800 nM, more preferably between 150 and 500 nM, even more preferably between 200 and 350 nM; in some embodiments about 236 nM, in some embodiments about 294 nM, in some embodiments about 460 nM, in some embodiments about 570 nM, in some embodiments about 603 nM, in some embodiments about 763 nM, in some embodiments about 982 nM;
capable to specifically bind to a tumour cell, wherein the tumour cell is characterized by being double-positive for PD-L1 and EGFR;
inhibits the dimerization of EGFR, but not the binding of EGF to EGFR;
inhibits EGFR-pathway signalling at least 2-fold or more, preferably between 3-fold and 10-fold, preferably between 3-fold and 8-fold, preferably between 3-fold and 5-fold; in some embodiments the IC₅₀ for inhibiting the EGFR-pathway is between 1 and 10 nm, preferably about 6.7 nM.
inhibits the binding of PD-L1 to PD-1 with an EC₅₀ of between 1 and 1000 nM, preferably of between 5 and 500 nM, preferably of between 50 and 250 nM, preferably of about 214 nM;
binds to double-positive cells (such as A431-cells) with EGFR and PD-L1 with “high affinity”, i.e. an “effective concentration of binding” an EC₅₀ of between 0.1 and 50 nM, of between 0.5 and 20 nM, preferably between 1 and 10 nM, preferably between 1.25 and 5 nM, preferably of about 1.37 nM;
binds to EGFR-positive cells with an EC₅₀ of between 5 and 50 nM, of between 10 and 20 nM, preferably of about 17.83 nM.
being further modified to an antibody-drug conjugate (ADC) by being conjugated to a therapeutic agent, such as drug, a tumour-growth inhibitor, an apoptotic signal molecule, a toxin or the like;
being further modified to extend its half-life by albumin-fusion, polyaminoacid fusion, Fc-fusion, or a polymer-conjugation with polyethylene glycol, sialic acid, starch and/or hyaluronic acid.

The half maximal inhibitory concentration (IC₅₀) is a measure of the potency of a substance in inhibiting a specific biological or biochemical function. IC₅₀ is a quantitative measure that indicates how much of a particular inhibitory substance (e.g., the inventive antibody) is needed to inhibit, in vitro, a given biological process or biological component by 50%. The biological component could be an enzyme, cell, cell receptor or microorganism, in the particular case of this invention it is EGFR and PD-L1-activity. IC₅₀ values are typically expressed as molar concentration.

IC₅₀ is commonly used as a measure of antagonist drug potency in pharmacological research. IC₅₀ is comparable to other measures of potency, such as EC₅₀ for excitatory drugs. EC₅₀ represents the dose or plasma concentration required for obtaining 50% of a maximum effect in vivo.

IC₅₀ can be determined with functional assays or with competition binding assays. In general, the Two-in-One antibody is loaded onto BLI biosensors and association is measured against the antigen, pre-incubated with its ligand or receptor. In the case that the antibody can still bind to the antigen despite binding of the natural ligand/receptor, it does not inhibit the interaction of the antigen with its ligand/receptor. If no binding is observed, the antibody binds an epitope that overlaps with the ligand/receptor (see also example section and FIGS. 4A and 5A).

In another aspect of the present invention variants based on the inventive Two-in-One antibody HCP-LCE were produced. First of all, a one-armed (oa) variant of the inventive antibody was produced to show its binding abilities and usefulness for further applications, such as scFv or Fab fragments.

Further, as also depicted in FIG. 7 and further described in the examples, five different trispecific variants were designed (cf. FIG. 7, “V1”-“V5”):
1. A “Knob-into-Hole” (KiH) variant (cf. FIG. 7, “V1”) consisting of HCP-LCE on one side and the heavy chain of the CD16 binder (NKE14), paired with the HCP-LCE light chain.
2. Two variants with two Fabs (see KiH variant) each side in both possible orientations (cf. FIG. 7, “V2” and “V4”):
   ICI2_outer and NKE14_inner
   NKE14_outer and ICI2_inner
3. both variants were also designed and produced as one-armed (oa) variants (cf. FIG. 7, “V3” and “V5”).

Thus, the present invention also pertains to trivalent and/or multivalent variants, which comprise besides binding-sites to EGFR and PD-L1 at least one further binding-site, able to bind for example to CD16.

In one aspect, further variants of the inventive antibodies were generated: A Knob-into-Hole (KiH) variant consisting of HCP-LCE on one side and a novel CD16 binder (HC16) on the other side, paired with the HCP-LCE light chain (as in FIG. 7, “V1”) Furthermore, two variants with two Fabs (see KiH variant) each side in both possible orientations were generated:
ICI2_outer and HC16_inner (as in FIG. 7, “V2”)
HC16_outer and ICI2_inner (as in FIG. 7, “V4”)
   both variants were also designed and produced as one-armed (oa) variants.

The ICI2_outer- and HC16_inner-variants showed the best binding profile.

In one further aspect, the present invention pertains to attenuated trispecific T-cell engager (TCE) antibodies, which are combined with an anti-interleukin 6 receptor (IL-6R) binding moiety in order to modulate cytokine activity (TriTECM). Overshooting cytokine release, culminating in cytokine release syndrome (CRS), is one of the severest adverse effects observed with T-cell immunotherapies, where the IL-6/IL-6R axis is known to play a pivotal role. By targeting two tumour-associated antigens, epidermal growth factor receptor (EGFR) and programmed death ligand 1 (PD-L1), simultaneously with a bispecific two-in-one antibody, high tumour selectivity together with checkpoint inhibition is achieved. In one embodiment tetrafunctional molecules that contained additional CD3- and IL-6R-binding modules are generated. These antibodies are able to compete with ligands for both PD-L1 and IL-6R as well as inhibit both EGF- and IL-6-mediated signalling pathways was observed. Furthermore, TriTECM molecules are able to activate T cells and trigger T-cell-mediated cytotoxicity through CD3-binding in an attenuated fashion. A decrease in pro-inflammatory cytokine interferon γ (IFNγ) after T-cell activation is observed for the TriTECM molecules compared to their respective controls lacking IL-6R binding, hinting at a successful attenuation and potential modulation via IL-6R. As IL-6 is a key player in cytokine release syndrome as well as being implicated in enhancing tumour progression, therefore such molecule designs reduce side effects and cytotoxicity observed with previous TCEs and widen their therapeutic windows.

In brief, the inventive tetrafunctional TriTECMs consist of:
i) dual tumour-targeting for increased tumour specificity by targeting EGFR and PD-L1,
ii) engagement of T cells via CD3E binding and bridging T cells in close proximity to tumour cells for tumour cell lysis,
iii) increased T-cell-mediated cytotoxicity by blocking the PD-L1/PD-1 axis (checkpoint inhibitors), and
iv) modulating cytokine storms or CRS by inhibiting the IL-6/IL-6R pathway by an anti-IL-6R scFv (FIG. 6).

While combination therapies with anti-IL-6R agents (tocilizumab, sarilumab) have been investigated and approved for therapeutics-induced cytokine release syndrome, simultaneous administration by being fused to the mAb itself according to the present invention might prove beneficial. Hypothetical clinical findings after CAR T-cell therapy report dramatic increases in cytokine levels 3 days after infusion, with tocilizumab having to be administered at day 6 or 7.

By using a full-length IgG1 backbone, the serum half-life of such a molecule would be approximately 21 days, thus being in frame with the current management approaches or interventions required when grade 2 CRS is reached. Furthermore, the direct fusion of an anti-IL-6R that blocks further downstream signalling by inhibiting the JAK/STAT pathway responsible for the promotion of tumour growth and metastasis provides beneficial anti-tumour properties. Such a synergistic effect is promoted by direct fusion to tumour-targeting antibodies rather than co-administration, thereby enhancing the accumulation of an IL-6R blocking entity in the tumour microenvironment. More details on these inventive antibodies can be derived from the example section (example 11).

Altogether, the advantages of TriTECMs can be used for novel biologicals for cancer therapies by harnessing the power of T cells (cf. FIG. 20).

The present invention therefore includes the adaptation of other CD3-binding antibody fragments as well as already-existing antibodies which were shown to be toxic due to CRS or other side-effects and might be recycled in the inventive format due to the attenuation and modulation of both T-cell activation and IL-6-mediated signalling. Due to the attenuation of the CD3-binding moiety, bivalent binding to CD3 is also part of this invention. Additionally, pentafunctional molecules are part of this invention by using an Fc that induces effector functions on cytokine release and T-cell-mediated cytotoxicity.

Alternative and/or further targets may be selected from the group consisting of cluster of differentiation 155 (CD155), also known as the poliovirus receptor, MHC class I chain-related proteins A and B (MicA/MicB), cluster of differentiation 159 (CD159), a NKG2 receptor for natural killer cells (NK cells) (there are 7 NKG2 types: A, B, C, D, E, F and H; NKG2D is an activating receptor on the NK cell surface, NKG2A dimerizes with CD94 to make an inhibitory receptor), cluster of differentiation 276 (CD276) or B7 homolog 3 (B7-H3), hepatitis A virus cellular receptor 2 (HAVCR2), also known as T-cell immunoglobulin and mucin-domain containing-3 (TIM-3), T-cell immuno-receptor with Ig and ITIM domains (TIGIT), B-cell maturation antigen (BCMA or BCM), also known as tumour necrosis factor receptor superfamily member 17 (TNFRSF17), interleukin-3 receptor (CD123), natural cytotoxicity triggering receptor 3 (NKp30) or (CD337), natural cytotoxicity triggering receptor 3 (NKp44) or (CD336), natural cytotoxicity triggering receptor 3 (NKp46) or (CD335), cluster of differentiation 27 (CD27), cluster of differentiation 96 (CD96) or Tactile (T-cell activation, increased late expression), NKRP1A, cluster of differentiation 355 (CD355) or Cytotoxic and regulatory T-cell molecule (CRTAM), and lymphocyte function-associated antigen 1 (LFA-1), and/or any combination thereof.

The binding kinetics as measured by BLI for the third or further target site should be in the range of between 5 nM and 1000 nM, preferably between 50 nM and 800 nM, more preferably between 100 nM and 750 nM, even more preferably between 200 nM and 500 nM, even more preferably between 250 nM and 390 nM, in some embodiments between 270-280 nM or about 277 nM, between 295-310 nM or about 302 nM, between 335-352 nM or about 342 nM, between 375-392 nM or about 382 nM.

In further embodiments, the present invention pertains to an isolated nucleic acid molecule encoding the bispecific monoclonal antibody or antigen-binding fragment or a substantially similar variant thereof.

In further embodiments, the present invention pertains to an expression vector comprising the before mentioned nucleic acid molecule.

In yet another aspect the invention pertains to a method of producing the antibody according to the invention, preferably a HCP-LCE- or HCE-LCP-antibody, or antigen-binding fragment or a substantially similar variant thereof comprising the steps of
i) Selecting a first and a second human target protein, preferably represented on the surface of tumour cells, preferably EGFR and PD-L1;
ii) Creating a first yeast surface display (YSD) library, comprising the following steps:
   a. Immunizing an animal of avian species, preferably a chicken, with the first target protein;
   b. Isolating variable heavy chain (VH) genes from cDNA derived from said immunized animal of avian species;
   c. Inserting the VH genes into a vector designed for expression, secretion, and display of proteins on the extracellular surface of Saccharomyces cerevisiae cells, preferably pYD1-derived, and transform yeast cells with said vector; in one embodiment said vector further comprises a constant heavy chain (CH1), glutamine synthetase (GS) and/or Aga2p (from Saccharomyces cerevisiae).
   d. Inserting genes for a common variable light chain (VL) (SEQ ID No.: 16) into an expression vector designed for expression, secretion, and display of proteins on the extracellular surface of Saccharomyces cerevisiae cells, preferably pYD1-derived and transform yeast cells with said vector; in one embodiment said vector further comprises a constant light chain (CL1);
   e. Combining the two yeast cells by yeast cell mating, resulting in diploid yeast cells displaying VH domains from the PD-L1-immunized animal of avian species, paired with the common variable light chain (VL) (SEQ ID No.: 16);
   f. Screening by fluorescence activated cell sorting (FACS) for the binding to the first target protein and isolate yeast cells displaying antibodies which specifically bind to the first target protein;
iii) Creating a second yeast surface display (YSD) library, comprising the following steps:
   a. Immunizing an animal of avian species, preferably a chicken, with the second target protein, preferably EGFR if the first antigen was PD-L1; or PD-L1 if the first antigen was EGFR;
   b. Isolating variable light chain (VL) genes from cDNA derived from said immunized animal of avian species;
   c. Inserting the common variable heavy chain (VH) genes (SEQ ID No.: 15) as described in the previous screen into a vector designed for expression, secretion, and display of proteins on the extracellular surface of Saccharomyces cerevisiae cells, preferably pYD1-derived, and transform yeast cells with said vector;
iv) Combining the yeast cells from the first YSD-library carrying heavy chains (VH) specifically binding to the first target protein with the second YSD-library carrying the light chains (VL) from the animal of avian species immunized with the second target protein, by yeast mating
v) Screening the resulting yeast cells by fluorescence activated cell sorting (FACS) for binding to both target proteins and/or inhibition of each of the two target proteins;
vi) Identifying the yeast cells comprising antibodies comprising heavy chains specifically binding to the first target protein and comprising light chains specifically binding to the second target protein;
vii) Isolating the bispecific, tetravalent monoclonal antibody or antigen-binding fragment binding to both target proteins (in one embodiment humanized and/or human), preferably EGFR and PD-L1.

Since each arm of the inventive antibodies, as produced by the before mentioned method, binds to both targets with at least medium affinity, it may be combined with another “arm” binding to at least one, at least two or more further targets. This is also exemplified in the example section, where the HCP-LCE-antibody according to the invention was combined with a CD16-binding entity (NKE14). Thus, this invention also encompasses methods to produce antibodies binding to one, two or more targets besides binding to EGFR and PD-L1.

The described method relies on screening of combinational immune-libraries and obviates the need for any additional CDR engineering as described in the prior art. Thus, this invention paves the way for further development of therapeutic antibodies derived from avian immunization with novel and tailor-made binding properties.

Thus, one advantage of the inventive method is that it allows for the isolation of chicken-derived Two-in-One antibodies without CDR engineering by combining the heavy chain of an anti-PD-L1 common light chain antibody with an anti-EGFR immune light chain library and YSD screening. The resulting antibody HCP-LCE simultaneously targets EGFR and PD-L1 at the same Fv fragment while exhibiting favourable biophysical properties and aggregation behaviour. The Two-in-One antibody can inhibit EGFR signalling by binding to dimerization domain II and to block the PD-1/PD-L1 interaction. Furthermore, HCP-LCE demonstrated specific cellular binding properties on EGFR/PD-L1 double-positive tumour cells. The inventive antibodies are the first Two-in-One antibodies without CDR engineering that simultaneously target two antigens with the same Fab fragment.

The invention further provides therapeutic compositions comprising the antibodies or antigen-binding fragments thereof of the present invention. The administration of therapeutic compositions in accordance with the invention will be administered with suitable carriers, excipients, and other agents that are incorporated into formulations to provide improved transfer, delivery, tolerance, and the like.

A multitude of appropriate formulations can be found in the formulary known to all pharmaceutical chemists: Remington's Pharmaceutical Sciences, Mack Publishing Company, Easton, PA.

These formulations include, for example, aqueous solutions, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax.

The dose may vary depending upon the age and the size of a subject to be administered, severity of the target disease, individual condition of the subject (for example in case of a kidney-disease the dosage may be reduced to between 25%-75% as compared to the dosage for healthy patients of the same gender and weight), route of administration, and the like.

Depending on the severity of the condition, the frequency and the duration of the treatment can be adjusted.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis.

Methods of introduction include, but are not limited to, intradermal, intramuscular, intraperitoneal, intravenous, subcutaneous, intranasal, epidural, inhalative and oral routes; for example, as full-length, fragment, FcRn, EPO-Fc, RNA- or DNA-precursor, or the like.

The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

The pharmaceutical composition can be also delivered in a vesicle, in particular a liposome. In certain embodiments, the pharmaceutical composition can be delivered in a controlled release system, such as a pre-filled syringe, a dermal patch, infusion (for example in case of Bi-specific T-cell engager- (BiTE)-antibodies), pen- or auto-injector-device (single-dose, multi-dose, disposable, re-usable), syrette, large-volume pump-device, and the like.

In one embodiment a pre-filled syringe, or auto-injector, or pump device is preferred.

In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracutaneous and intramuscular injections, drip infusions, etc.

These injectable preparations may be prepared by methods publicly known.

For example, the injectable preparations may be prepared, e.g., by dissolving, suspending or emulsifying the antibody or its salt described above in a sterile aqueous medium or an oily medium conventionally used for injections.

As the aqueous solution for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a non-ionic surfactant [e.g., polysorbate 80, HCO-50 (polyoxyethylene (50 mol) adduct of hydrogenated castor oil)], etc.

As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

A pharmaceutical composition of the present invention can be delivered subcutaneously or intravenously with a standard needle and syringe.

In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused.

In a disposable pen delivery device, there is no replaceable cartridge. Rather, the disposable pen delivery device comes prefilled with the pharmaceutical composition held in a reservoir within the device.

Once the reservoir is emptied of the composition, the entire device is discarded.

Numerous reusable pens and auto-injection delivery devices have applications in the subcutaneous delivery of a pharmaceutical composition of the present invention. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN 70/30™ pen (Eli Lilly and Co., Indianapolis, IN), NOVOPEN™ I, II and Ill (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™ OPTIPEN STARLET™, and OPTICLIK™ (sanofi-aventis, Frankfurt, Germany), to name only a few.

Examples of disposable pen delivery devices having applications in subcutaneous delivery of a pharmaceutical composition of the present invention include, but certainly are not limited to the SOLOSTAR™ pen (sanofi-aventis), the FLEXPEN™ (Novo Nordisk), and the KWIKPEN™ (Eli Lilly).

Advantageously, the pharmaceutical compositions for oral or parenteral use described above are prepared into dosage forms in a unit dose suited to fit a dose of the active ingredients.

Such dosage forms in a unit dose include, for example, tablets, pills, capsules, injections (ampoules), suppositories, etc.

The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

In further embodiments, the present invention pertains to a bispecific monoclonal antibody or antigen-binding fragment, or a substantially similar variant thereof for the use in the identification, prevention and/or treatment of a disease or condition related to cancer in a patient.

In particular the inventive antibodies can be used for identification, prevention and/or treatment of PD-L1-positive tumours, such as non-small cell lung carcinomas, malignant melanomas, bladder carcinomas, Hodgkin's lymphomas, kidney cancer, glioblastoma, and breast cancer. High levels of PD-L1 are often found in these, as well as some other types of cancer and can be treated effectively with immunotherapy using the inventive antibodies.

The invention provides therapeutic methods in which the antibody or antibody fragment of the invention is useful to treat above mentioned cancers. The antibodies or antigen-binding fragments of the invention are particularly useful for combination therapies for example with histone deacetylase (HDAC) inhibitors (HDIs). HDIs have a long history of use in psychiatry and neurology as mood stabilizers and anti-epileptics. More recently they are being investigated as possible treatments for cancers and inflammatory diseases.

The “classical” HDIs act exclusively on Class I, ∥ and Class IV HDACs by binding to the zinc-containing catalytic domain of the HDACs. These classical HDIs can be classified into several groupings named according to the chemical moiety that binds to the zinc ion (except cyclic tetrapeptides which bind to the zinc ion with a thiol group). Some examples in decreasing order of the typical zinc binding affinity:
1. hydroxamic acids (or hydroxamates), such as trichostatin A,
2. cyclic tetrapeptides (such as trapoxin B), and the depsipeptides,
3. benzamides,
4. electrophilic ketones, and
5. the aliphatic acid compounds such as phenylbutyrate and valproic acid.

“Second-generation” HDIs include the hydroxamic acids vorinostat (SAHA), belinostat (PXD101), LAQ824, and panobinostat (LBH589); and the benzamides: entinostat (MS-275), tacedinaline (C1994), and mocetinostat (MGCD0103).

The sirtuin Class Ill HDACs are dependent on NAD+ and are, therefore, inhibited by nicotinamide, as well as derivatives of NAD, dihydrocoumarin, naphthopyranone, and 2-hydroxynaphthaldehydes.

The antibodies or antigen-binding fragments of the invention are particularly useful for combination therapies with one or more of any agent selected from the group consisting of: chemotherapy drugs and/or cytostatic drugs of any kind, such as for example alkylating agents, antimetabolites, anthracyclines, taxanes, vinca alkaloids. Specific substances used in such a treatment may be for example cyclophosphamide, methotrexate, 5-fluoruracil, vincristin, prednisolone, epirubicin, cisplatin, folinic acid, irinotecan, oxaliplatin, mitomycin C, etoposid, bleomycin, procarbazin, lomustin, adriamycin, and/or capecitabin.

In one embodiment entinostat, also known as SNDX-275 and MS-275, a benzamide histone deacetylase inhibitor undergoing clinical trials for treatment of various cancers, is used.

In one embodiment the inventive bispecific monoclonal antibody or antigen-binding fragment or a substantially similar variant thereof can be used to from an antibody-drug-conjugate (ADC). Antibody-drug conjugates or ADCs are a class of biopharmaceutical drugs designed as a targeted therapy for treating cancer. Unlike chemotherapy, ADCs are intended to target and kill tumor cells while sparing healthy cells.

ADCs combine the targeting capabilities of monoclonal antibodies with the cancer-killing ability of cytotoxic drugs. They can be designed to discriminate between healthy and diseased tissue.

In one embodiment an anti-cancer drug is coupled to the inventive antibodies that specifically targets tumours displaying EGFR and PD-L1 on their surface. The biochemical reaction between the antibody and the target protein (antigen) triggers a signal in the tumour cell, which then absorbs or internalizes the antibody together with the linked cytotoxin. After the ADC is internalized, the cytotoxin kills the cancer. This targeting limits side effects and gives a wider therapeutic window than other chemotherapeutic agents.

The drug may be a cytotoxic payload selected from the group consisting of mertansine (DM1), duocarmycin (DUO), auristatin F-hydroxypropylamide (AF-HPA), monomethylauristatin F (MMAF), pyrrolobenzodiazepines (PBDs), monomethyl auristatin E (MMAE), calicheamicin (calich.), ravtansine (DM4), active metabolite of irinotecan (SN-38), novel cytotoxic dolastatin 10 analogue (PF06380101), DX-8951 a derivative of the camptothecin analog exatecan (DXd), doxorubicin (DOX), and auristatin-0101 (Aur0101) and/or any combination thereof.

The antibody may be connected to the drug directly or via a linker. A stable ADC linker ensures that less of the cytotoxic payload falls off before reaching a tumour cell, improving safety, and limiting dosages.

Linkers are based on chemical motifs including disulphides, hydrazones or peptides (cleavable), or thioethers (noncleavable). Cleavable and noncleavable linkers were proved to be safe in preclinical and clinical trials. For example, “brentuximab vedotin” includes an enzyme-sensitive cleavable linker that delivers the antimicrotubule agent monomethyl auristatin E or MMAE, a synthetic antineoplastic agent, to human-specific CD30-positive malignant cells. MMAE inhibits cell division by blocking the polymerization of tubulin. Because of its high toxicity MMAE cannot be used as a single-agent chemotherapeutic drug. However, MMAE linked to the antibodies of the invention may be used for a specific tumour-cell targeted approach.

Another example “trastuzumab emtansine” is a combination of the microtubule-formation inhibitor mertansine (DM-1) and antibody trastuzumab that employs a stable, non-cleavable linker.

The availability of better and more stable linkers has changed the function of the chemical bond. The type of linker, cleavable or noncleavable, lends specific properties to the cytotoxic drug. For example, a non-cleavable linker keeps the drug within the cell. As a result, the entire antibody, linker and cytotoxic (anti-cancer) agent enter the targeted cancer cell where the antibody is degraded into an amino acid. The resulting complex—amino acid, linker and cytotoxic agent—is considered to be the active drug. In contrast, cleavable linkers are detached by enzymes in the cancer cell. The cytotoxic payload can then escape from the targeted cell and, in a process called “bystander killing”, attack neighbouring cells.

Another type of cleavable linker, currently in development, adds an extra molecule between the cytotoxin and the cleavage site. This allows researchers to create ADCs with more flexibility without changing cleavage kinetics. Researchers are developing a new method of peptide cleavage based on Edman degradation, a method of sequencing amino acids in a peptide. Also under development are site-specific conjugation (TDCs) and novel conjugation techniques to further improve stability and therapeutic index, a emitting immunoconjugates, antibody-conjugated nanoparticles and antibody-oligonucleotide conjugates.

In another aspect of the present invention the inventive antibodies may be combined with further binding sites for other targets in order to increase the specificity of the antibody even further. This is possible due to the specific nature of the antibodies presented therein which can bind to EGFR/PD-L1 with just one Fab-region and/or one antibody-arm only. In case of scFv-fragments one could imagine the fusion to a second or further scFv-fragment able to bind at least one other target, creating trivalent, tetravalent or multivalent constructs. Obviously also other combinations with other antigen-binding proteins could be envisaged, for example scFv-fragment-ligand combination, scFv-fragment-anticaline-combination and many more (for example other small modular immunopharmaceuticals (SMIPs)).

In another aspect of the present invention the inventive antibodies are used in chimeric antigen receptor-therapy (CAR-T-cell-therapy). The inventive antibodies may serve as one of the chimeric antigen-receptors, or may be used in combination in order to improve the specificity of the modified T-cells.

In another aspect of the present invention the inventive antibodies may be used as sensors in a device or binding entity in an assay, e.g., an ELISA in order to stratify patients and/or patient groups before, during and/or after undergoing therapy.

In yet another aspect the invention pertains to a monoclonal antibody or antigen-binding fragment, or a substantially similar variant thereof characterized in that at least one Fab-region binds specifically to epidermal growth factor receptor (EGFR) and programmed cell death-ligand 1 (PD-L1).

In a further aspect the invention pertains to a monoclonal antibody or antigen-binding fragment, or a substantially similar variant thereof characterized in that at least one Fab-region binds specifically to epidermal growth factor receptor (EGFR) and programmed cell death-ligand 1 (PD-L1) further comprising an element binding to cluster of differentiation 3 (CD3), preferably CD3ε, and/or to interleukin 6 (IL-6) and/or interleukin 6-receptor (IL-6R).

In a further aspect the invention pertains to the antibody or antigen-binding fragment, or a substantially similar variant thereof as described hereinunder, comprising at least one Fv-region binding specifically to epidermal growth factor receptor (EGFR) and programmed cell death-ligand 1 (PD-L1).

In a further aspect the invention pertains to the antibody or antigen-binding fragment or a substantially similar variant thereof as described hereinunder, comprising at least one Fv-region binding specifically to epidermal growth factor receptor (EGFR) and programmed cell death-ligand 1 (PD-L1); and comprising at least a second Fv-region binding specifically to specifically cluster of differentiation 3 (CD3), preferably CD3ε, and/or to interleukin 6 (IL-6) and/or interleukin 6-receptor (IL-6R).

In a further aspect the invention pertains to the antibody or antigen-binding fragment, or a substantially similar variant thereof as described hereinunder, wherein the paratopes, i.e., the CDR-regions binding to EGFR and PD-L1, on said antibody or antigen-binding fragment or a substantially similar variant thereof do not overlap.

In a further aspect the invention pertains to the antibody or antigen-binding fragment, or a substantially similar variant thereof as described hereinunder, wherein the paratopes, i.e. the CDR-regions binding to EGFR and PD-L1 and/or CD3, preferably CD3ε, and/or IL-6 and/or IL-6R, on said antibody or antigen-binding fragment or a substantially similar variant thereof do not overlap.

In a further aspect the invention pertains to the antibody or antigen-binding fragment, or a substantially similar variant thereof as described hereinunder, is capable of inhibiting EGFR-signalling and blocking the PD-1/PD-L1 interaction.

In a further aspect the invention pertains to the antibody or antigen-binding fragment, or a substantially similar variant thereof as described hereinunder, is capable of inhibiting EGFR-signalling and blocking the PD-1/PD-L1 interaction, and/or the CD3, preferably the CD3ε-engager element binds specifically to CD3, or preferably CD3ε, and/or blocks and/or inhibits the IL6-IL-6R-gp130 pathway.

In a further aspect the invention pertains to the antibody or antigen-binding fragment, or a substantially similar variant thereof as described hereinunder, wherein the EGFR binding is at the dimerization domain II of EGFR and wherein the antibody prevents binding of PD-L1 to PD-1 and/or competes for binding to PD-L1 with durvalumab.

In a further aspect the invention pertains to the antibody or antigen-binding fragment, or a substantially similar variant thereof as described hereinunder, comprising
a heavy chain CDR1 (H-CDR1) domain with an amino acid sequence selected from the group consisting of SEQ ID No.: 03; and/or SEQ ID No.: 33;
a heavy chain CDR2 (H-CDR2) domain with an amino acid sequence selected from the group consisting of SEQ ID No.: 04; and/or SEQ ID No.: 34;
a heavy chain CDR3 (H-CDR3) domain with an amino acid sequence selected from the group consisting of SEQ ID No.: 05; and/or SEQ ID No.: 35;
   and/or
a light chain CDR1 (L-CDR1) domain with an amino acid sequence selected from the group consisting of SEQ ID No.: 06; and/or
a light chain CDR2 (L-CDR2) domain with an amino acid sequence selected from the group consisting of SEQ ID No.: 07; and/or
a light chain CDR3 (L-CDR3) domain with an amino acid sequence selected from the group consisting of SEQ ID No.: 08.

In a further aspect the invention pertains to antibody or antigen-binding fragment, or a substantially similar variant thereof as described hereinunder, comprising
at least 95% identical to a heavy chain VH-region with an amino acid sequence selected from the group consisting of SEQ ID No.: 01; and/or SEQ ID No.: 28, and/or SEQ ID No.: 30;
and/or
at least 95% identical to a light chain VL-region domain with an amino acid sequence selected from the group consisting of SEQ ID No.: 02, and/or SEQ ID No.: 29, and/or SEQ ID No.: 31;

In a further aspect the invention pertains to antibody or antigen-binding fragment, or a substantially similar variant thereof as described hereinunder, comprising an amino acid sequence identical to the amino acid sequence of SEQ ID No.: 01 and/or SEQ ID No.: 28, and/or SEQ ID No.: 30; and of SEQ ID No.: 02; and/or SEQ ID No.: 29, and/or SEQ ID No.: 31.

In a further aspect the invention pertains to the antibody or antigen-binding fragment, or a substantially similar variant thereof as described hereinunder, comprising a heavy chain variable region of the amino acid sequence in SEQ ID No.: 01 and a light chain variable region of the amino acid sequence in SEQ ID No.: 02.

In a further aspect the invention pertains to the antibody or antigen-binding fragment, or a substantially similar variant thereof as described hereinunder, comprising a heavy chain variable region of the amino acid sequence in SEQ ID No.: 28 and a light chain variable region of the amino acid sequence in SEQ ID No.: 29.

In a further aspect the invention pertains to the antibody or antigen-binding fragment, or a substantially similar variant thereof as described hereinunder, comprising a heavy chain variable region of the amino acid sequence in SEQ ID No.: 30 and a light chain variable region of the amino acid sequence in SEQ ID No.: 31.

In a further aspect the invention pertains to the antibody or antigen-binding fragment, or a substantially similar variant thereof as described hereinunder, further characterized by one or more of the following:
human or humanized, preferably grafted into a human IgG1-scaffold;
also capable of binding to at least at least a further target protein selected from the group consisting of Fcγ-receptor IIIa/IIIb (CD16a/b), cluster of differentiation 155 (CD155), also known as the poliovirus receptor, MHC class I chain-related proteins A and B (MicA/MicB), cluster of differentiation 159 (CD159), a NKG2 receptor for natural killer cells (NK cells) (there are 7 NKG2 types: A, B, C, D, E, F and H; NKG2D is an activating receptor on the NK cell surface, NKG2A dimerizes with CD94 to make an inhibitory receptor), cluster of differentiation 276 (CD276) or B7 homolog 3 (B7-H3), hepatitis A virus cellular receptor 2 (HAVCR2), also known as T-cell immunoglobulin and mucin-domain containing-3 (TIM-3), T-cell immuno-receptor with Ig and ITIM domains (TIGIT), B-cell maturation antigen (BCMA or BCM), also known as tumor necrosis factor receptor superfamily member 17 (TNFRSF17), interleukin-3 receptor (CD123), natural cytotoxicity triggering receptor 3 (NKp30) or (CD337), natural cytotoxicity triggering receptor 3 (NKp44) or (CD336), natural cytotoxicity triggering receptor 3 (NKp46) or (CD335), cluster of differentiation 27 (CD27), cluster of differentiation 96 (CD96) or Tactile (T-cell activation, increased late expression), NKRP1A, cluster of differentiation 355 (CD355) or Cytotoxic and regulatory T-cell molecule (CRTAM), and lymphocyte function-associated antigen 1 (LFA-1), and/or any combination thereof;
capable of binding specifically to PD-L1-protein with an affinity (KD) of between 50 and 500 nM;
capable of binding specifically to EGFR-protein with an affinity (KD) of 150 and 1000 nM;
capable to specifically bind to a tumour cell, wherein the tumour cell is characterized by being double-positive for PD-L1 and EGFR;
inhibits the dimerization of EGFR, but not the binding of EGF to EGFR;
inhibits EGFR-pathway signalling at least 2-fold or more;
inhibits the binding of PD-L1 to PD-1 with an EC50 of between 5 and 500 nM;
binds to double-positive cells with EGFR and PD-L1 with an EC50 of between 0.5 and 20 nM;
being further modified to an antibody-drug conjugate (ADC) by being conjugated to a therapeutic agent, such as drug, a tumour-growth inhibitor, an apoptotic signal molecule, a toxin or the like;
being further modified to extend its half-life by albumin-fusion, polyaminoacid fusion, fc fusion, or a polymer-conjugation with polyethylene glycol, sialic acid, starch and/or hyaluronic acid.

In a further aspect the invention pertains also to an isolated nucleic acid molecule encoding the monoclonal antibody or antigen-binding fragment, or a substantially similar variant thereof according to the present invention.

In a further aspect the invention pertains also to an expression vector comprising the nucleic acid molecule as described before.

In a further aspect the invention pertains also to a method of producing the antibody or antigen-binding fragment or a substantially similar variant thereof comprising the steps of
i. Selecting a first and a second human target protein, preferably represented on the surface of tumour cells, preferably EGFR and PD-L1;
ii. Creating a first yeast surface display (YSD) library, comprising the following steps:
   a. Immunize an animal of avian species, preferably a chicken, with the first target protein;
   b. Isolate variable heavy chain (VH) genes from cDNA derived from said immunized animal of avian species;
   c. Insert the VH genes into a vector designed for expression, secretion, and display of proteins on the extracellular surface of Saccharomyces cerevisiae cells, preferably pYD1-derived, and transform yeast cells with said vector;
   d. Insert genes for a common variable light chain (VL) (SEQ ID No.: 16) into an expression vector designed for expression, secretion, and display of proteins on the extracellular surface of Saccharomyces cerevisiae cells, preferably pYD1-derived and transform yeast cells with said vector;
   e. Combine the two yeast cells by yeast cell mating, resulting in diploid yeast cells displaying VH domains from the PD-L1-immunized animal of avian species, paired with the common variable light chain (VL) (SEQ ID No.: 16);
   f. Screen by fluorescence activated cell sorting (FACS) for the binding to the first target protein and isolate yeast cells displaying antibodies which specifically bind to the first target protein;
iii. Creating a second yeast surface display (YSD) library, comprising the following steps:
   a. Immunize an animal of avian species, preferably a chicken, with the second target protein; preferably EGFR if the first antigen was PD-L1, or PD-L1 if the first antigen was EGFR;
   b. Isolate variable light chain (VL) genes from cDNA derived from said immunized animal of avian species;
   c. Insert genes for the common variable heavy chain (VH) gene (SEQ ID No.: 15) into a vector designed for expression, secretion, and display of proteins on the extracellular surface of Saccharomyces cerevisiae cells, preferably pYD1-derived, and transform yeast cells with said vector;
iv. Combining the yeast cells from the first YSD-library carrying heavy chains (VH) specifically binding to the first target protein with the second YSD-library carrying the light chains (VL) from the animal of avian species immunized with the second target protein, by yeast mating
v. Screening the resulting yeast cells by fluorescence activated cell sorting (FACS) for binding to both target proteins; and/or inhibition of each of the two target proteins;
vi. Identifying the yeast cells comprising antibodies comprising heavy chains specifically binding to the first target protein and comprising light chains specifically binding to the second target protein;
vii. Isolating the bispecific, tetravalent humanized or a human monoclonal antibody or antigen-binding fragment binding to both target proteins, preferably EGFR and PD-L1,
viii. Receiving the antibody or antigen-binding fragment or a substantially similar variant thereof according to any one of the previous claims.

In a further aspect the invention pertains also to a pharmaceutical composition comprising the monoclonal antibody or antigen-binding fragment, or a substantially similar variant thereof as described hereinunder, and a pharmaceutically acceptable carrier.

In a further aspect the invention pertains also to a monoclonal antibody or antigen-binding fragment, or a substantially similar variant thereof as described hereinunder for the use in the identification, prevention and/or treatment of a disease or condition in a patient. In one aspect the inventive antibody further avoids and/or reduces any hypercytokinemia, also called a “cytokine storm”, that is a physiological reaction in humans and other animals in which the innate immune system causes an uncontrolled and excessive release of pro-inflammatory signalling molecules called cytokines.

### SHORT DESCRIPTION OF THE FIGURES

FIG. 1: Schematic representation demonstrating the generation of the Two-in-One antibody HCP-LCE.

The anti-PD-L1 antibody ICI2 is derived from a common light chain YSD library (SEQ ID No.: 16). The VH fragment of the anti-PD-L1 antibody ICI2 was paired with an anti-EGFR VL library by yeast mating. FACS screening and subsequent reformatting into the full-length antibody format enabled the isolation and production of a Two-in-One antibody targeting PD-L1 and EGFR. Created with BioRender.com.

FIG. 2: YSD-based EGFR epitope mapping of HCP-LCE.
(A) Schematic representation of EGFR extracellular domains I to IV and the six EGFR fragments investigated in this study. (B) Binding of HCP-LCE to yeast cells expressing different truncated EGFR fragments was detected using the goat anti-human Fc PE antibody. Measurements without HCP-LCE served as secondary control. HCP-LCE binds to EGFR fragment 1-294.

FIG. 3: Characterization of antigen binding of the Two-in-One antibody HCP-LCE by BLI-measurements.
(A) BLI-measurements of HCP-LCE, ICI2_H2 and LCE against EGFR and PD-L1. HCP-LCE binds both antigens, whereas ICI2_H2 exclusively targets PD-L1. LCE shows no binding to either antigen. (B) BLI-assisted simultaneous binding assay. The one-armed HCP-LCE variant was loaded onto biosensors and antigens are added stepwise, revealing simultaneous EGFR and PD-L1 binding.

FIG. 4: EGF competition and EGFR signaling assays.
(A) BLI-assisted EGF competition assay. HCP-LCE was loaded onto AHC biosensors and subsequently associated to EGFR pre-incubated with varying EGF concentrations. HCP-LCE binds to EGFR despite EGF binding. (B) Cell-based EGFR signalling assay. EGF-induced AKT phosphorylation was analysed in EGFR-positive A549 cells. SEB7, ICI2_H2, the bispecific construct SEB7×ICI2_H2, cetuximab and durvalumab were tested in comparison to the Two-in-One antibody HCP-LCE. All measurements were performed in triplicates.

FIG. 5: PD-1 competition and PD-1/PD-L1 blockage assays.
(A) BLI-assisted PD-1 competition assay. HCP-LCE was loaded onto FAB2G-biosensors and subsequently associated to PD-L1 pre-incubated with varying PD-1 concentrations. The binding of HCP-LCE to PD-L1 at different PD-1 concentrations reveals dose-dependent binding. (B) Cell-based PD-1/PD-L1 blockage assay. SEB7, ICI2_H2, the bispecific construct SEB7×ICI2_H2 and durvalumab were tested in comparison to the Two-in-One antibody HCP-LCE. EC₅₀ values: durvalumab, 0.39 nM; ICI2_H2, 8.60 nM; SEB7×ICI2_H2, 179 nM; HCP-LCE, 214 nM. Luciferase activity is plotted against the logarithmic antibody concentration. All measurements were performed in duplicates, and the experiments were repeated at least three times, yielding similar results.

FIG. 6: Cell binding of EGFR and PD-L1 on A431 cells.

Cell titration of HCP-LCE, ICI2_H2, SEB7 and the bispecific construct on EGFR/PD-L1 double positive A431 cells. A variable slope four-parameter fit was utilized to fit the resulting curves. EC₅₀ values: HCP-LCE, 1.37 nM; SEB7, 3.92 nM; SEB7×ICI2_H2, 2.83 nM. All measurements were performed in triplicates, and the experiments were repeated at least three times, yielding similar results.

FIG. 7: Trispecific variants based on the Two-in-One antibody HCP-LCE.

A schematic representation of the structure and arrangement of the trispecific antibody variants is depicted, as well as their corresponding one-armed variants.

FIG. 8: BLI-assisted simultaneous binding assay.

The Knob-into-hole antibody or the one-armed variants were loaded onto biosensors and antigens are added stepwise, revealing simultaneous PD-L1, EGFR and CD16 binding.

FIG. 9: BLI-assisted EGF competition assay.

The trispecific antibodies based on the Two-in-One antibody HCP-LCE were loaded onto AHC biosensors and subsequently associated to EGFR pre-incubated with varying EGF concentrations. The binding of the antibodies to EGFR at different EGF concentrations reveals dose-dependent binding.

FIG. 10: BLI-assisted PD-1 competition assay.

The trispecific antibodies based on the Two-in-One antibody HCP-LCE were loaded onto FAB2G-biosensors and subsequently associated to PD-L1 pre-incubated with varying PD-1 concentrations. The binding of the antibodies to PD-L1 at different PD-1 concentrations reveals dose-dependent binding.

FIG. 11: EGFR signaling inhibition.

The antibodies were tested for their capability to inhibit phospho-AKT using the A549 cell line. HTRF Ratio is shown on the y-axis, the antibody concentration is shown on the x-axis.

FIG. 12: Affinity measurements.

Binding kinetics of SEB7, SEB7×ICI2_H2 and oaHCP-LCE to EGFR and PD-L1.

FIG. 13: SDS-PAGE analysis.

SDS-PAGE analysis was done with ICI2_H2, SEB7, HCP-LCE, SEB7×ICI2_H2, oaHCP-LCE, LCE, KiH, ICI2_outer and NKE14_outer under reducing conditions revealing high purity and the expected molecular weights. SDS-PAGE analysis of KiH, ICI2_outer, NKE14_outer and HCP-LCE under non-reducing conditions revealing the expected molecular weights.

FIG. 14: Conceptual architecture of TriTECMs.

A) Molecular design of two TriTECM constructs, (αCD3ε)K(αIL-6R)+H(αIL-6R) as scFv(VH)-IgG1(H)-scFv2 and K(αCD3ε)+H(αIL-6R) as an IgG1(H)-scFv2. The dark scFv represents the anti-CD3 scFv (αCD3ε), the light scFv represents the anti-IL-6R scFv (αIL-6R), the Fabs originate from HCP-LCE and an effector-silenced IgG1 backbone was used. The circle between the CH3 domains represent the Knob-into-Hole technology for heterdimerisation. B) Control variants with the same coding of the Fab and scFv regions as in A.C) SDS-PAGE analysis under reducing (left) and non-reducing (right) conditions for all variants numbered as in A and B.

FIG. 15: Simultaneous binding to all targets.

A) Dot plots for simultaneous binding of Calcein AM-stained A431 cells (y-axis) and bound Jurkats (x-axis). Binding to Jurkats via CD3 was detected using anti-human Fc PE. Events in Q1 represent the Calcein AM-stained A431 cells (FITC+/PE−), while those in Q4 represent Jurkats without CD3-binding (FITC−/PE−). A shift towards Q2 (FITC+/PE+) depicts binding of the A431 cells after incubation with Jurkats, and Q3 shows CD3-binding on Jurkats (FITC−/PE+). 20′000 events are displayed per dot plot. B) Quantification of events in Q3 (FITC−/PE+) versus Q2 (FITC+/PE+) from A. Positive events in both quadrants ensures Jurkat & A431 engagement.

FIG. 16: BLI-based simultaneous binding assay.

Investigation of simultaneous binding via BLI by loading antibodies onto AHC biosensors and associated first to 200 nM IL-6R, followed by 500 nM of EGFR and PD-L1 sequentially for 300 seconds each.

FIG. 17: Ligand competition and signaling inhibition.

A) IL-6/IL-6R competition assay by BLI. B) PD-L1/PD-1 competition assay by BLI. A-B) The antibodies were loaded onto FAB2G biosensors and associated either to the receptor alone (dark lines) or pre-incubated with ligand (light lines). C-F) HTRF assays for signalling activation. A549 cells were pre-incubated with the indicated amounts of antibodies and then stimulated with the respective ligands. pSTAT3 was measured for IL-6-mediated signalling (C) or combination of EGF- and IL-6 stimulation (E). pAKT was measured for EGF-mediated signalling (D) or combination of EGF- and IL-6 stimulation (F). Boxed numbers represents the antibody variant with the following coding: 3—K+H; 7—K(αIL-6R)+H(αIL-6R); 5—(αCD3ε)K+H; 1—(αCD3ε)K(αIL-6R)+H(αIL-6R); 4—K(αCD3ε)+H; 2—K(αCD3ε)+H(αIL-6R).

FIG. 18: T-cell activation.

A-B) T-cell activation bioassay NFAT pathway with A431 as target cells. C) Dot plots for measurement of CD25-APC (x-axis) and CD69-FITC (y-axis) of PBMCs after 48 h incubation with 30 nM of the indicated antibodies. D-F) Quantification of different quadrants. D) Percentage of parent for Q1 (upper left) for CD2−/CD69+, E) Percentage of parent for Q2 (upper right) for CD25+/CD69+ events. F) Percentage of parent for Q3 (lower right) for CD25−/CD69+ events. Statistical significance from one-way ANOVA analysis: *<0.05, **<0.001, ***<0.0001 p-value.

FIG. 19: Cytokine release and T-cell-mediated cytotoxicity.

A) IFNγ secretion of PBMCs stimulated with the indicated construct and amount (in nM) for 72 h. B) Determination of CD25-FITC levels of PBMCs stimulated for 72 h via flow cytometry. One-way ANOVA revealed***<0.0001 p-value compared to the blank control. C-D) Dead cell determination by propidium iodide (PI) staining after 24 h co-incubation of A431 cells and PBMCs in an effector-to-target ratio of 10:1. E) Determination of T-cell-mediated cytotoxicity by LDH release of A431 cells after 48 h incubation with PBMCs and the indicated amounts of antibodies (10:1 effector-to-target ratio). A431 only represents the basal killing level.

FIG. 20: Schematic representation of the four functionalities of tetrafunctional TriTECM molecules, as exemplified by (αCD3ε)K(αIL-6R)+H(αIL-6R).

FIG. 21: ADCC-assay.

To investigate immune cell stimulation and efficiency of cell killing, the Promega ADCC luminescent reporter assay was used. As target cells, EGFR/PD-L1 double positive A431 cells were used. The HCP-LCE antibody containing a LALA mutation (filled squares) failed to mediate ADCC effects as expected. Antibody variants are represented as follows: circles—ICI2_outer/HC16_inner LALA; triangles— HC16/ICI2 KiH, outlined squares—HC16_outer/ICI2_inner LALA; hexagons— oaHC16_outer; stars— HCP-LCE with a wild-type Fc; filled squares— HCP-LCE with LALA mutation.

### SEQUENCES

In this application the following sequences are used:

SEQ ID No. 1: Inventive antibody (HCP-LCE), full variable heavy chain amino acid sequence.

SEQ ID No. 2: Inventive antibody (HCP-LCE), full variable light chain amino acid sequence.

SEQ ID No. 3: Inventive antibody (HCP-LCE), CDR-H1 amino acid sequence.

SEQ ID No. 4: Inventive antibody (HCP-LCE), CDR-H2 amino acid sequence.

SEQ ID No. 5: Inventive antibody (HCP-LCE), CDR-H3 amino acid sequence.

SEQ ID No. 6: Inventive antibody (HCP-LCE), CDR-L1 amino acid sequence.

SEQ ID No. 7: Inventive antibody (HCP-LCE), CDR-L2 amino acid sequence.

SEQ ID No. 8: Inventive antibody (HCP-LCE), CDR-L3 amino acid sequence.

SEQ ID No. 9: CD16-binding antibody (NKE14), full variable heavy chain amino acid sequence.

SEQ ID No. 10: CD16-binding antibody (NKE14), CDR-H1 amino acid sequence.

SEQ ID No. 11: CD16-binding antibody (NKE14), CDR-H2 amino acid sequence.

SEQ ID No. 12: CD16-binding antibody (NKE14), CDR-H3 amino acid sequence.

SEQ ID No. 13: Inventive antibody (HCP-LCE), full length amino acid sequence of the heavy chain.

SEQ ID No. 14: Inventive antibody (HCP-LCE), full length amino acid sequence of the light chain.

SEQ ID No. 15: Common variable heavy chain used in yeast display, amino acid sequence of the heavy chain.

SEQ ID No. 16: Common variable light chain used in yeast display, amino acid sequence of the light chain.

SEQ ID No. 17: Knob-into-hole (KiH) variant, amino acid sequence of the heavy chain, “knob”-version.

SEQ ID No. 18: Knob-into-hole (KiH) variant, amino acid sequence of the heavy chain, “hole”-version.

SEQ ID No. 19: Knob-into-hole (KiH) variant, amino acid sequence of the light chain.

SEQ ID No. 20: ICI2_outer & NKE14_inner variant, amino acid sequence of the heavy chain (full length).

SEQ ID No. 21: ICI2_outer & NKE14_inner variant, amino acid sequence of the light chain (full length).

SEQ ID No. 22: NKE14_outer & ICI2_inner variant, amino acid sequence of the heavy chain (full length).

SEQ ID No. 23: NKE14_outer & ICI2_inner variant, amino acid sequence of the light chain (full length).

SEQ ID No. 24: The one-armed variants (oa) all used the same light chain amino acid sequence (full length) depicted here.

SEQ ID No. 25: The one-armed variants (oa) all used the same heavy chain (“hole”-version) amino acid sequence (full length) depicted here.

SEQ ID No. 26: The one-armed variants (oa) ICI2_outer & NKE14_inner variant used the heavy chain (“knob”-version) amino acid sequence (full length) depicted here.

SEQ ID No. 27: The one-armed variants (oa) NKE14_outer & ICI2_inner variant used the heavy chain (“knob”-version) amino acid sequence (full length) depicted here.

SEQ ID No. 28: The heavy chain of the “full-length HC16_outer and ICI2_inner LALA”-construct as used in example 11.

SEQ ID No. 29: The light chain of the “full-length HC16_outer and ICI2_inner LALA”-construct as used in example 11.

SEQ ID No. 30: The heavy chain of the “full-length ICI2_outer and HC16_inner LALA”-construct as used in example 11.

SEQ ID No. 31: The light chain of the “full-length ICI2_outer and HC16_inner LALA”-construct as used in example 11.

SEQ ID No. 32: The HC16 full variable heavy chain as used in the constructs in in example 11.

SEQ ID No. 33: The CDR-H1 (that is the CDR 1 of the heavy chain) of HC16 as used in the constructs in in example 11.

SEQ ID No. 34: The CDR-H1 (that is the CDR 1 of the heavy chain) of HC16 as used in the constructs in in example 11.

SEQ ID No. 35: The CDR-H3 (that is the CDR 3 of the heavy chain) of HC16 as used in the constructs in in example 11.

SEQ ID No. 36: This sequence represents the (αCD3ε)K(αIL6R)+H(αIL6R)-construct, i.e. the full-length knob heavy chain part of it. This was used in example 12.

SEQ ID No. 37: This sequence represents the (αCD3ε)K(αIL6R)+H(αIL6R)-construct, i.e. the full-length hole heavy chain part of it. This was used in example 12.

SEQ ID No. 38: This sequence represents the K(αCD3ε)+H(αIL6R)-construct, i.e. the full-length knob heavy chain part of it. This was used in example 12.

SEQ ID No. 39: This sequence represents the K(αCD3ε)+H(αIL6R)-construct, i.e. the full-length hole heavy chain part of it. This was used in example 12.

SEQ ID No. 40: This sequence represents the αCD3E scFv—construct. This was used in example 12.

SEQ ID No. 41: This sequence represents the αIL-6R scFv—construct. This was used in example 12.

Further proteins are used hereinunder, which sequence and further information is available from the UniProt-database (https:www.uniprot.org/) (version of Feb. 2, 2021):
CD16: UniProtKB—P08637
PD-1: UniProtKB—Q15116
PD-L1: UniProtKB—Q9NZQ7
EGF: UniProtKB—P01133
EGFR: UniProtKB—P00533
Sarilumab: UniProtKB—P12318 (Drugbank Accession No. DB11767)
Tocilizumab: UniProtKB—AONOL5 (Drugbank Accession No. DB06273)
Foralumab: UniProtKB—P07766 (Drugbank Accession No. DB16170)

Furthermore, the antibody durvalumab (MED14736) is a commercially available antibody, which has been published for example in the National Library of Medicine (https://pubchem.ncbi.nlm.nih.gov/substance/249565666) (Version 11, Sep. 2, 2021).

The antibody cetuximab is a commercially available antibody, which has been published in the Kegg-library (hhttps:www.genome.ip/entry/D03455) (Version Feb. 28, 2022).

The antibody SEB7 has been published by Bogen et al., Front. Immunol., 23 Dec. 2020, https://doi.org/10.3389/fimmu.2020.606878.

### EXAMPLES

### Example 1: Chicken Library Generation and Screening for ICI2

Based on the spleen-derived RNA from a chicken (Gallus gallus) immunized with four doses of commercially available PD-L1-Fc, cDNA was synthesised and VH genes were amplified by PCR. VH-encoding PCR amplicons were subcloned into a pYD1-derived vector and subsequently transformed into EBY100 yeast cells. BJ5464 yeast cells harbouring a pYD1-derived vector encoding a common light chain were combined with these EBY100 cells by yeast mating. The resulting diploid library was sorted by FACS for two rounds for Fab display and binding to decreasing concentrations of PD-L1. In the third round, a competition staining with PD-L1 and PD-1 was performed in order to isolate binders against the interaction site of PD-L1 and PD-1. The sorting-outcome of the third round only exhibited antibodies binding PD-L1, in one embodiment with an overlapping epitope with durvalumab. All unique binders derived from this campaign were produced as mAbs. Subsequent BLI analysis revealed that ICI2 exhibits the highest affinity towards PD-L1 and was therefore chosen for further development.

### Example 2: Two-In-One Library Generation and Screening

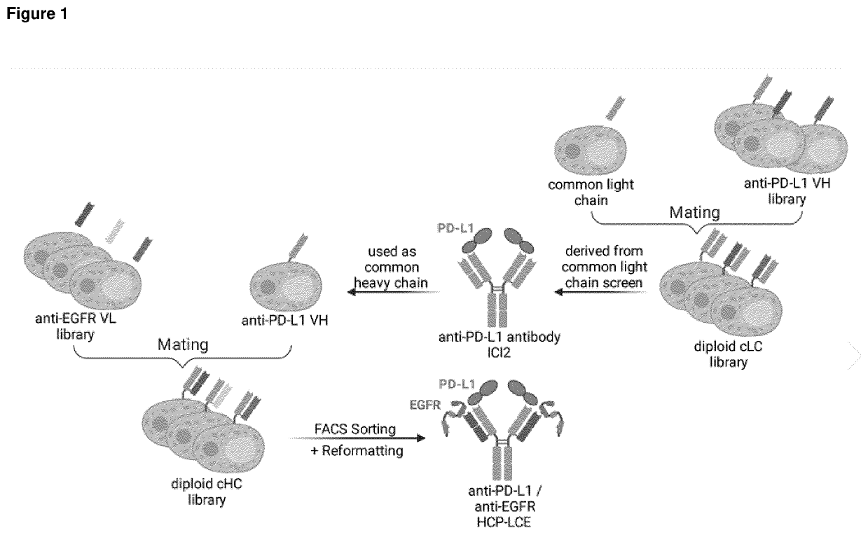

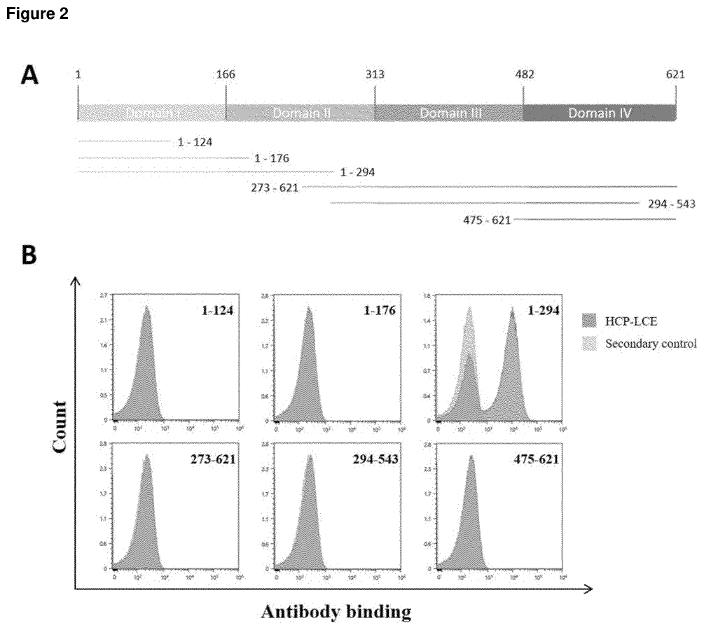

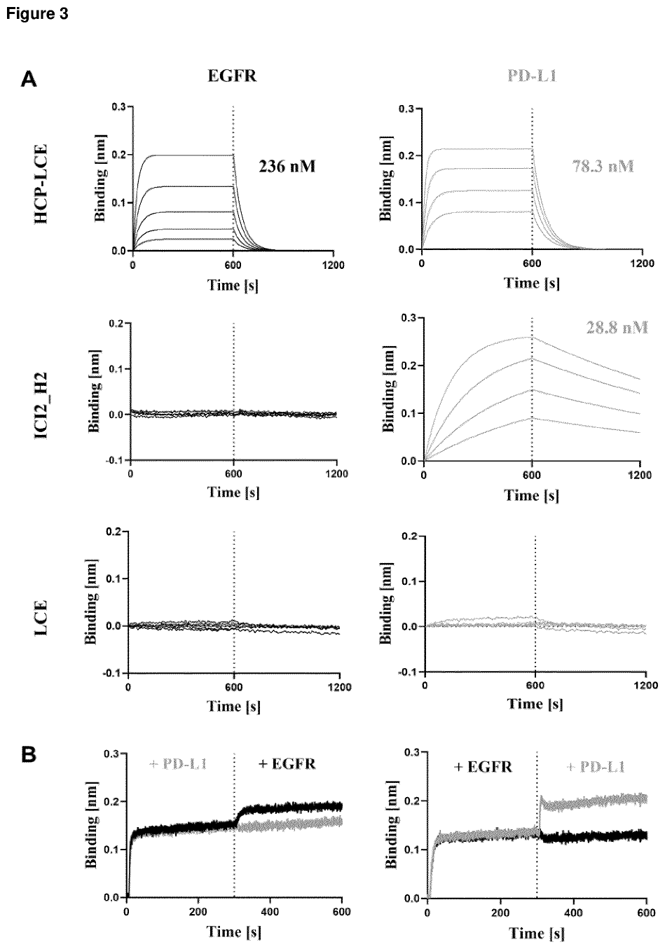

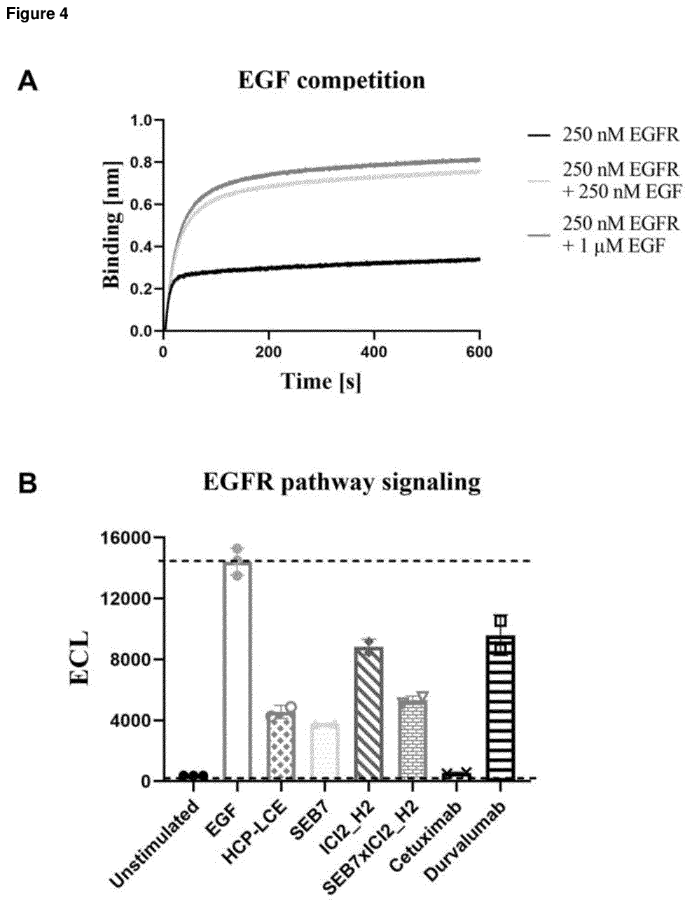

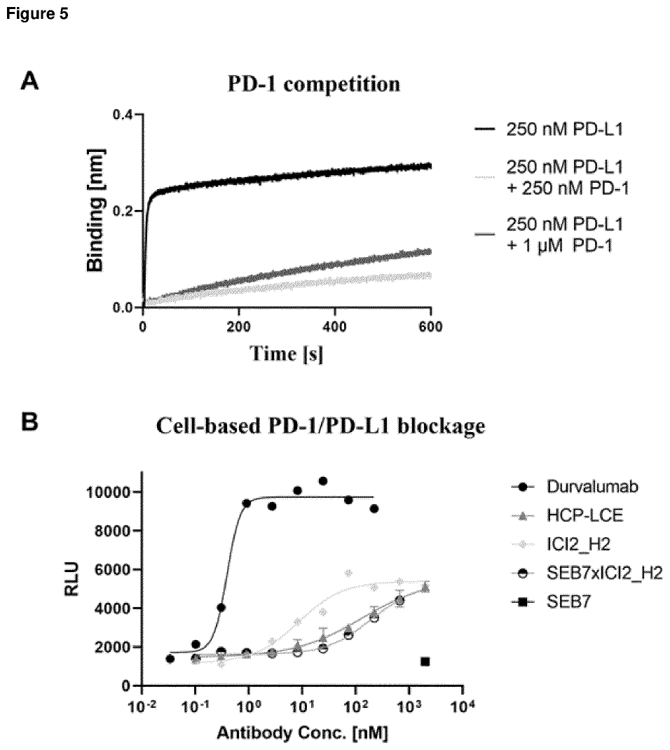

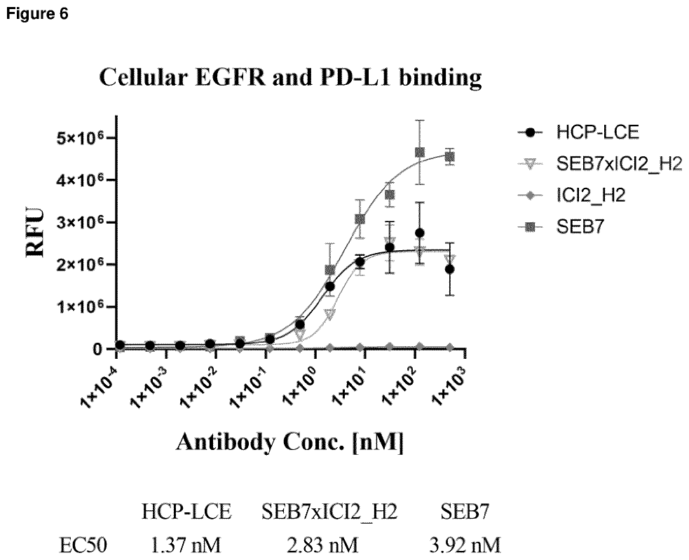

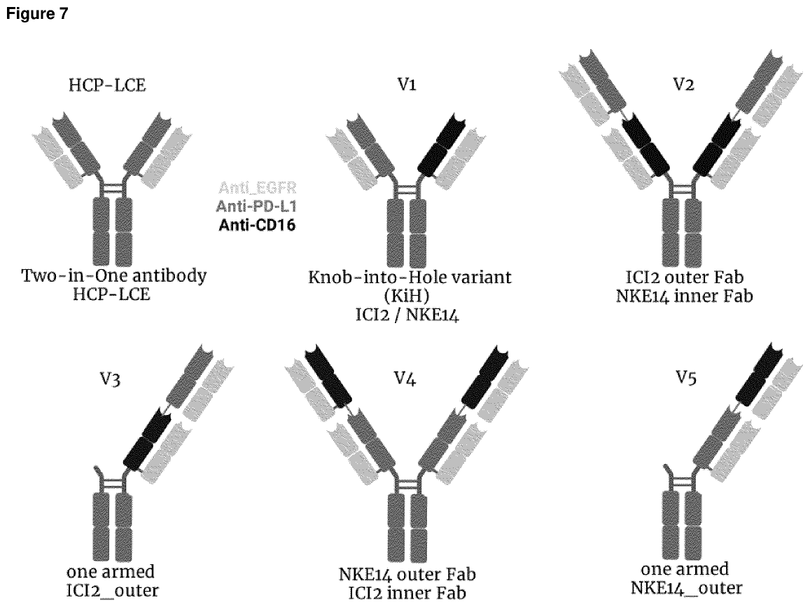

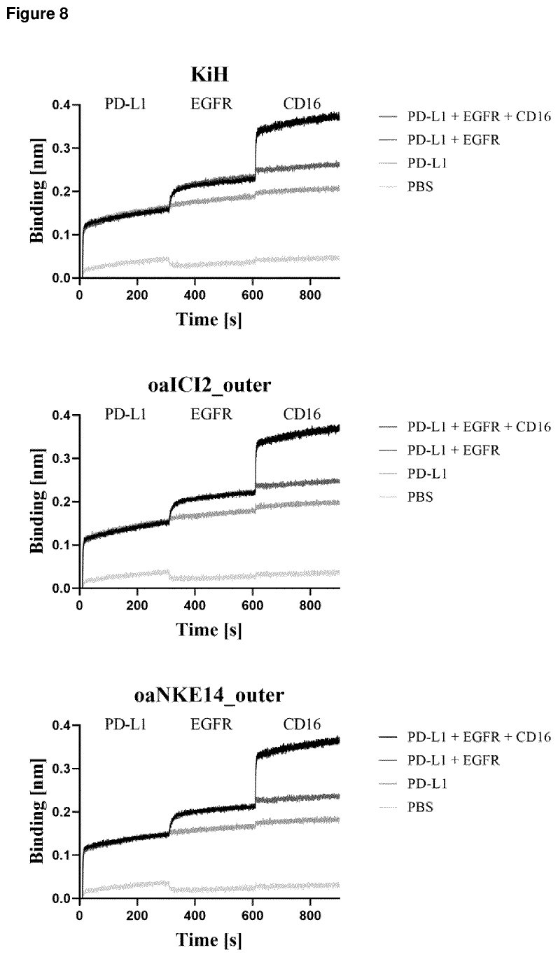

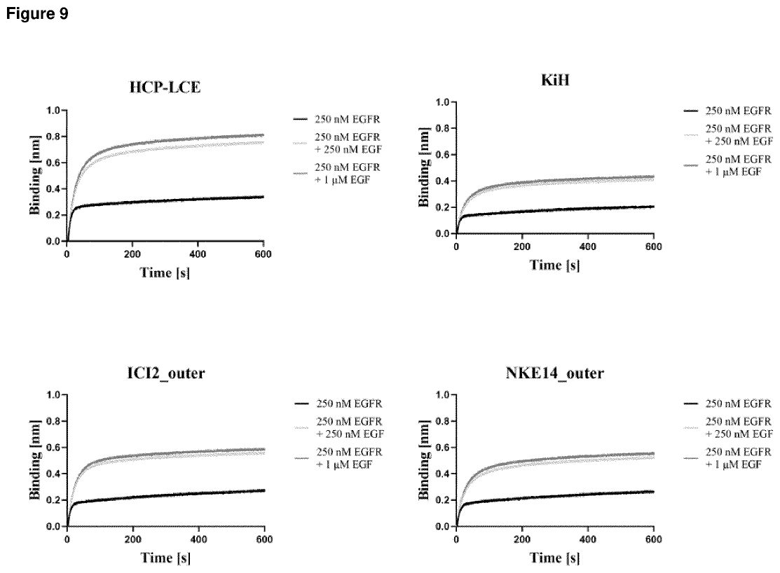

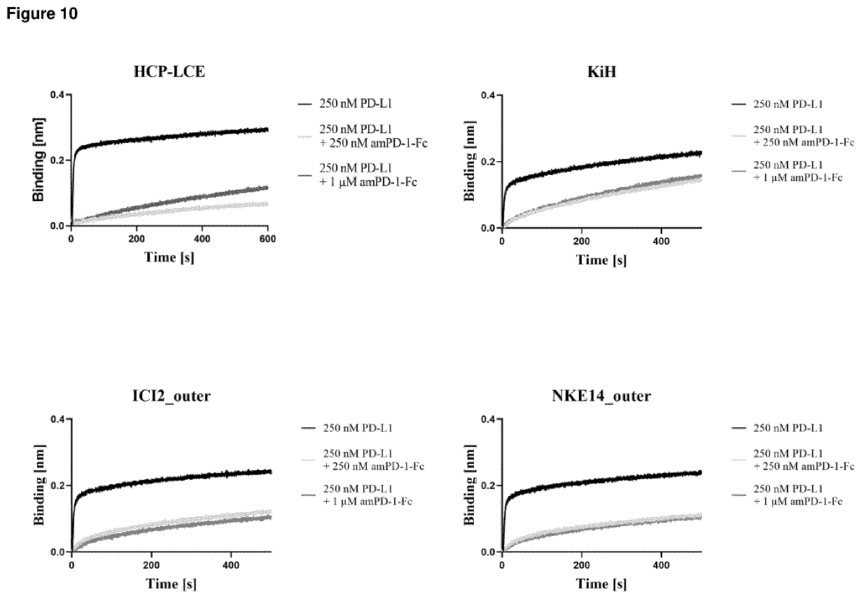

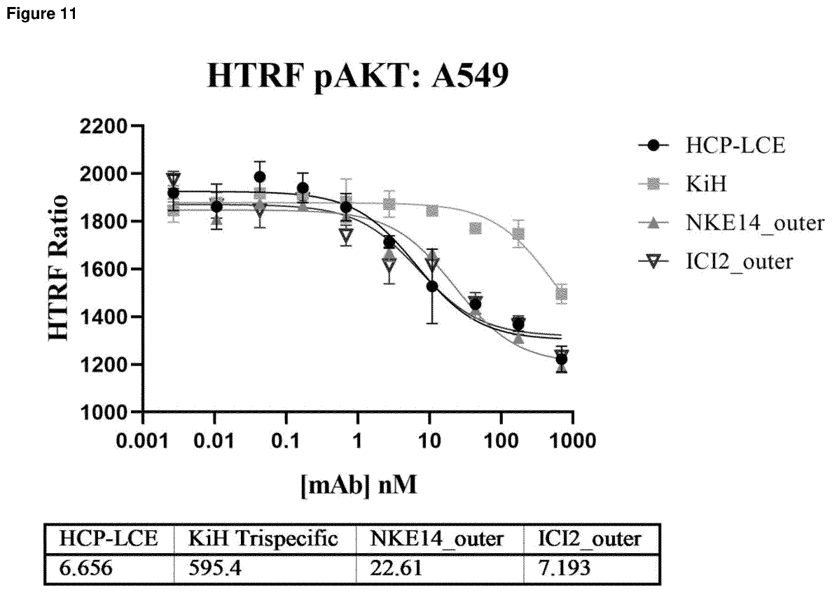

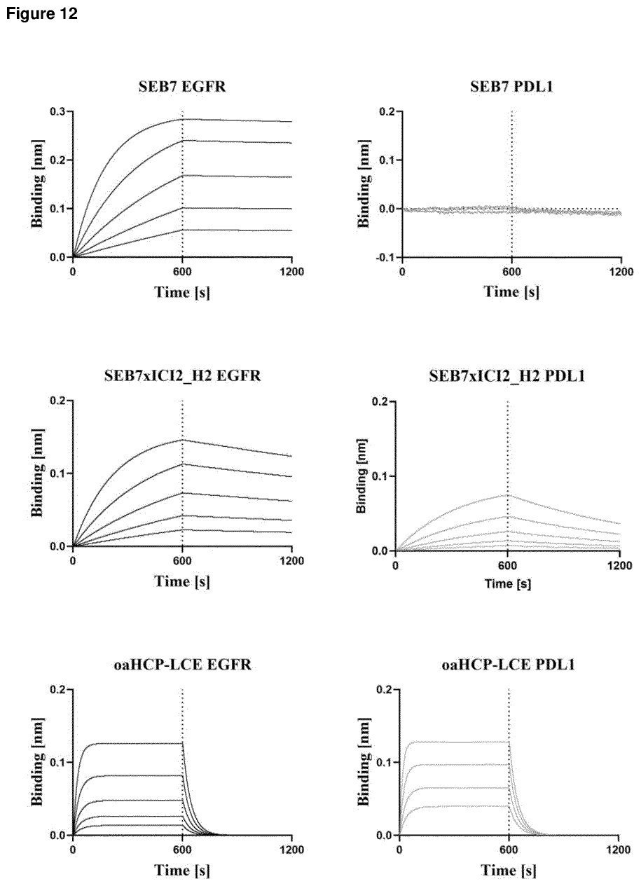

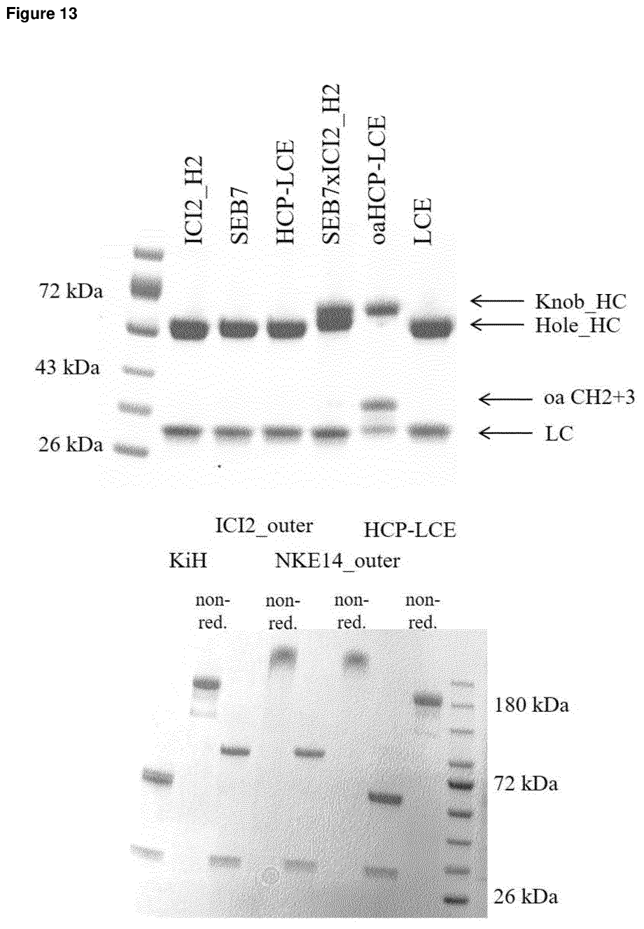

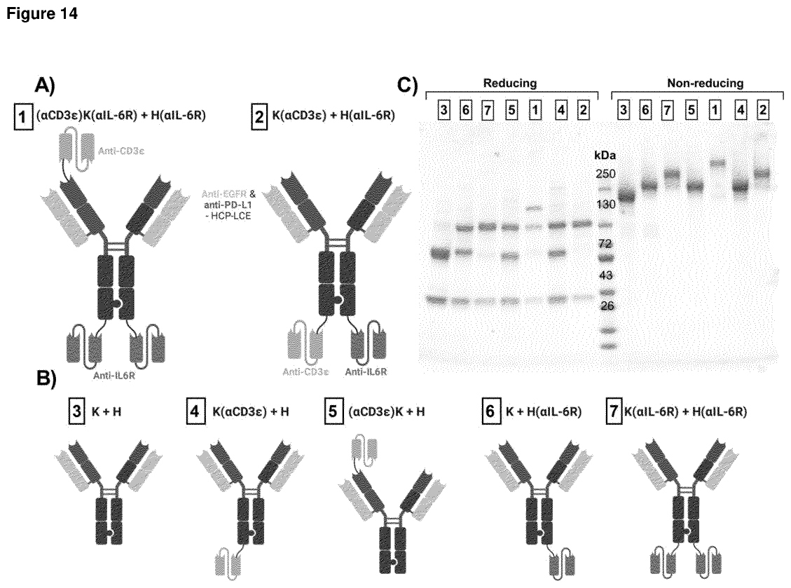

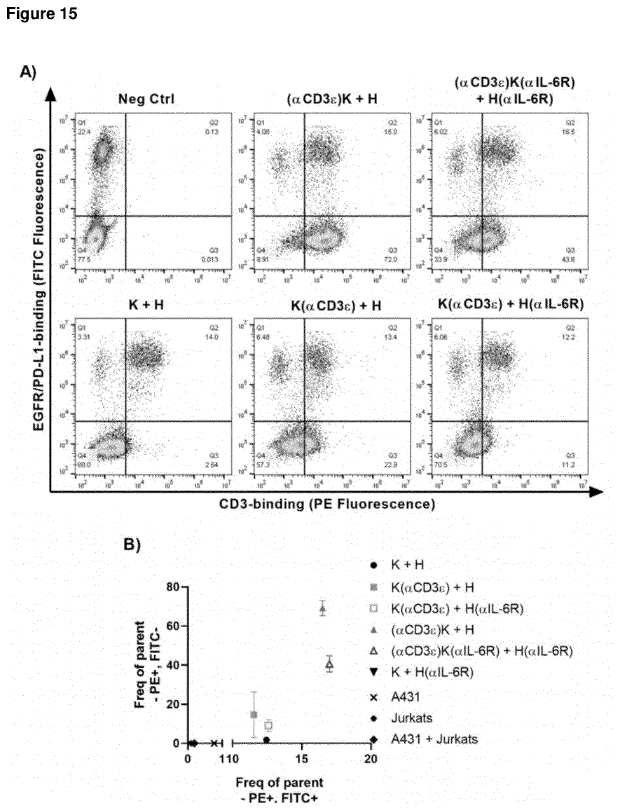

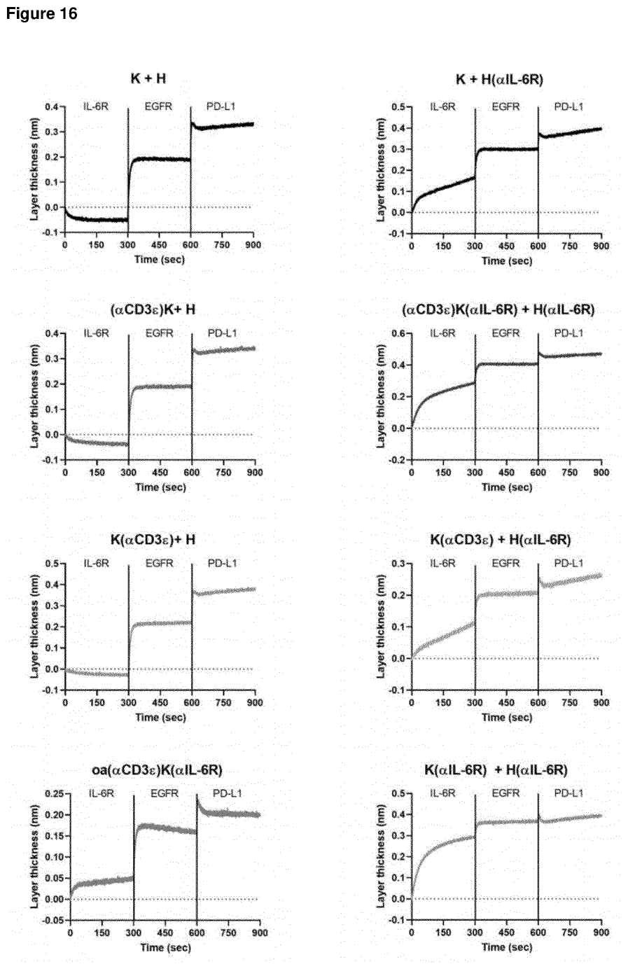

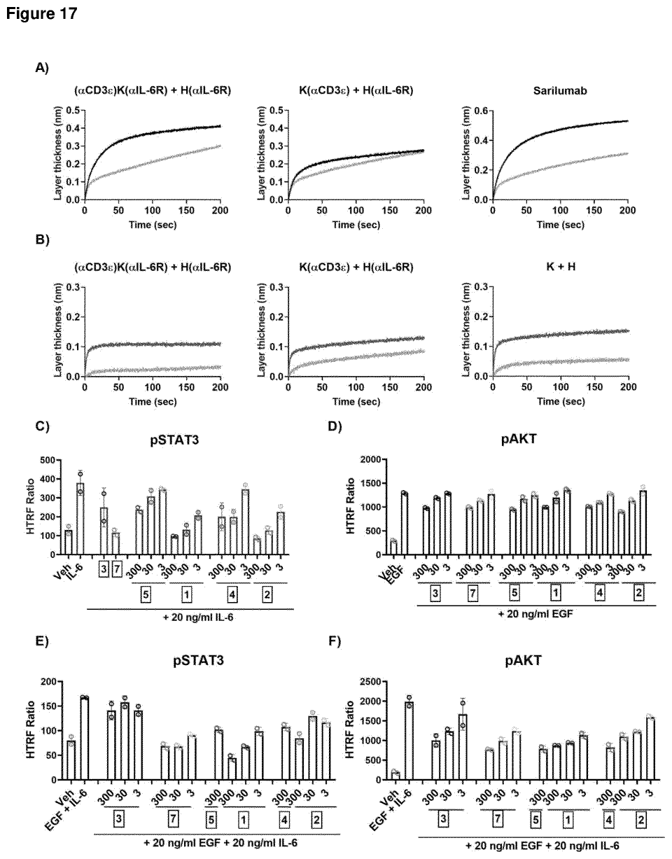

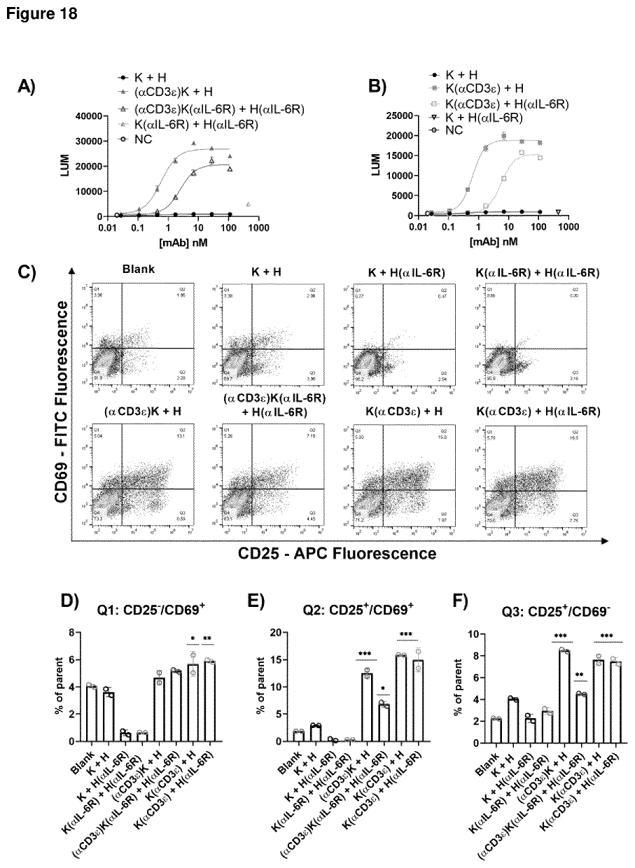

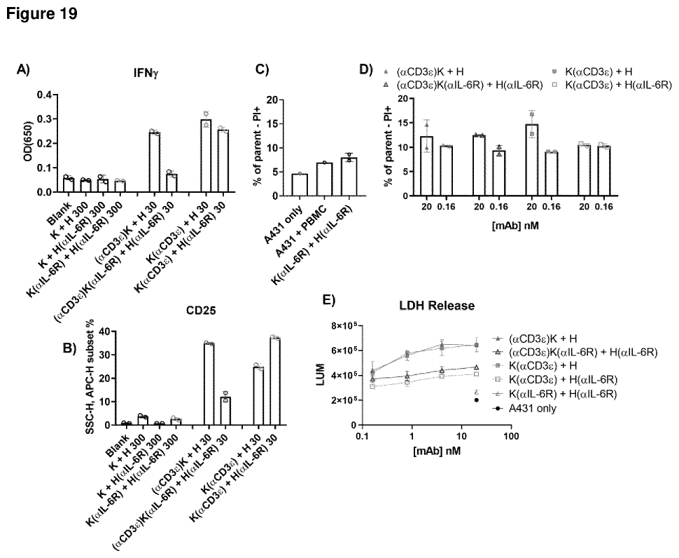

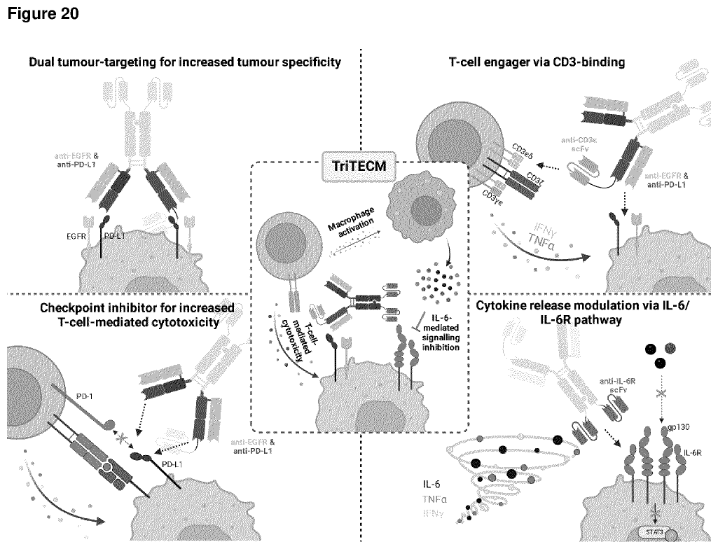

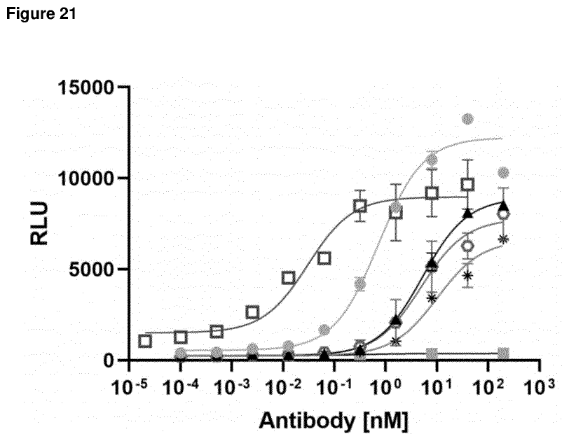

In order to generate a Two-in-One antibody, we sought to combine the heavy chain of a chicken-derived antibody with a chicken-derived immune YSD light chain library followed by subsequent screening for binding properties towards two antigens. As model targets, the extracellular domains of PD-L1 (PD-L1-ECD) and EGFR (EGFR-ECD) were chosen. Currently, various monoclonal antibodies targeting either EGFR, including the therapeutic antibodies panitumumab, necitumumab, nimotuzumab and cetuximab, or PD-L1, among them durvalumab, avelumab and atezolizumab are approved for tumor treatment in multiple countries. Recently, our group isolated a chicken-derived anti-PD-L1 antibody called ICI2. Since ICI2 exhibited a common light chain as it was utilized in a multispecific setup, we assumed that the heavy chain CDRs are mainly responsible for antigen recognition and could tolerate various light chains, as reported for other antibodies. In order to isolate a Two-in-One antibody targeting both PD-L1 and EGFR, the ICI2 heavy chain was paired with a diversity of anti-EGFR light chains (FIG. 1). The light chain library was generated by amplification of VL genes from cDNA derived from a chicken immunized with EGFR-ECD and subsequent insertion into a pYD1-derived vector encoding a human lambda CL by homologous recombination in BJ5464 yeast cells. The light chain diversity of 2.9×10⁸ transformants was combined with EBY100 yeast cells encoding the ICI2 VH-CH1 fragment by yeast mating (FIG. 1), resulting in adequate oversampling of the estimated light chain diversity, which is estimated to be about 5×10⁸ unique variants.

This diploid common heavy chain yeast library was screened by FACS over three consecutive sorting rounds with 250 nM EGFR-Fc. This yielded in the enrichment of a yeast population carrying the genes for Fab fragments recognizing both EGFR-Fc and PD-L1-Fc with 250 nM of the respective antigen. Fc binding could be excluded based on binding analysis to an unrelated Fc fusion protein. Sequence analysis of ten randomly chosen clones revealed one distinct VL sequence, which was enriched in the sorting process.

### Example 3: EGFR Epitope Mapping on the Subdomain Level

Expi293F cells were co-transformed using the isolated VL sequence reformatted into a pTT5-derived vector encoding a lambda CL sequence and a pTT5 vector encoding the ICI2 heavy chain. Purification of the Two-in-One antibody HCP-LCE (heavy chain PD-L1—light chain EGFR), was performed using Protein A affinity chromatography.

The extracellular region of EGFR consists of two homologous ligand-binding domains (domains I and III) and two cystine-rich domains (domains II and IV). The binding of EGF to the EGFR monomers at domains I and Ill promotes domain rearrangement to expose the dimerization arm in domain II finally resulting in the generation of EGFR homodimers. For full EGFR activation, ligand binding and EGFR dimerization are crucial.

To analyse which of the four extracellular EGFR domains is targeted by HCP-LCE, flow cytometric analysis was performed using yeast cells displaying truncated fragments of the EGFR-ECD. Since HCP-LCE exclusively targets EGFR fragment 1-294 but neither 1-124 nor 1-176 (FIG. 2), it was mapped to EGFR domain II, which is involved in receptor dimerization. Cetuximab is known to target domain III inhibiting EGF-binding to EGFR, while matuzumab blocks the receptor activation by sterically preventing the domain rearrangement. Binding of HCP-LCE to EGFR domain II may suggest inhibition of receptor dimerization and subsequent activation.

### Example 4: Affinity Measurement

BLI measurements were performed in order to determine the affinity of HCP-LCE to PD-L1 and EGFR (cf. FIG. 3). In addition, the affinity of a one-armed HCP-LCE variant (oaHCP-LCE) was determined. Therefore, the Knob-into-Hole technology was utilized to ensure heterodimerization of the heavy chains of oaHCP-LCE. A His-Tag C-terminally fused to the Hole-Fc and a TwinStrep-Tag C-terminally cloned to the Knob-Fc allowed two-step purification by immobilized metal affinity chromatography (IMAC), followed by StrepTactin XT chromatography. Furthermore, affinity measurements of the HCP-LCE heavy chain combined with an unrelated light chain (ICI2_H2) and that of the HCP-LCE light chain together with an unrelated heavy chain (LCE) to the two proteins of interest were performed.

HCP-LCE was able to bind both antigens with a KD of 78.3 nM and 236 nM for PD-L1 and EGFR binding, respectively, exhibiting a high off-rate (FIG. 3A, as well as Table 2 in example 8 below). The one-armed variant showed slightly lower affinities which might presumably be caused by the lower avidity (FIG. 12). Variant ICI2_H2 exclusively targeted PD-L1 with affinity in the double digit nanomolar range, whereas variant LCE showed no binding to either PD-L1 or EGFR (FIG. 3A, as well as Table 2 in example 8 below). This suggests that only the three HCP-LCE heavy chain CDRs are responsible for PD-L1 binding, contrary to EGFR binding involving overlapping heavy and light chain CDRs.

Since EGFR and PD-L1 are widely expressed on healthy cells, simultaneous binding of both proteins is essential to increase tumour-specificity. To analyze the binding behaviour of HCP-LCE and to verify whether both antigens can be targeted simultaneously with a single Fab, oaHCP-LCE was loaded onto AHC biosensors and sequentially incubated with both target proteins of interest. It is essential to use the one-armed variant, since the symmetric HCP-LCE antibody could target one antigen with each Fab fragment. Binding to PD-L1 first and EGFR second as well as reverse binding was considered. The oaHCP-LCE variant was able to bind both antigens simultaneously, regardless of the order of target protein incubation (FIG. 3B). These findings indicate that the paratopes do not overlap.

### Example 5: Biophysical Characterization

Size-exclusion chromatography (SEC) profiles demonstrated that HCP-LCE exhibited favourable properties with almost no measurable aggregation (Table 2). Monospecific antibodies ICI2_H2 and LCE showed an excellent aggregation profile, while the bispecific EGFR- and PD-L1-binding antibody SEB7×ICI2_H2 and the oaHCP-LCE variant exhibited aggregation of 10.8% and 11.20%, respectively (Table 2). Retention times were as expected (Table 2), indicating the accurate size of the antibodies produced, which is in accordance with SDS-PAGE analysis (FIG. 13). In the case of oaHCP-LCE and SEB7×ICI2_H2, SDS-PAGE demonstrated the expression of similar amounts of both heavy chains, with the TwinStrep-tagged Fc having a significantly higher molecular weight than the His-tagged Fc (FIG. 13).

Thermal stability was analysed using NanoDSF, yielding T_{M} values between 58.9° C. and 67.3° C., indicating high thermal stability of all variants (Table 2). For full-length antibodies, two to three T_{M} values are expected based on unfolding of the Fab fragment and CH2/CH3 domains. The lowest T_{M} value was utilized to compare the stability of all antibodies generated.

### Example 6: EGF and PD-1 Competition

To investigate antibody-mediated ligand receptor blockade, a competition assay was performed by aid of biolayer interferometry (cf. FIGS. 4 & 5). For analysis of EGF competition, Anti-human IgG Fc Capture (AHC) biosensors were loaded with HCP-LCE and subsequently associated to 250 nM EGFR pre-incubated with 250 nM or 1000 nM EGF. Due to the binding of EGFR to EGF, the complex exhibits a larger molecular size compared to EGFR alone. Binding of HCP-LCE to this complex, therefore, results in a higher increase in layer thickness compared to binding of EGFR alone (FIG. 4A), indicating that the antibody does not target the interaction site of EGF and EGFR, which is consistent with the YSD-based epitope mapping experiment (FIG. 2B). EGF binds simultaneously to EGFR domains I and Ill, whereas HCP-LCE targets EGFR domain II, which is involved in receptor dimerization.

For total EGFR activation, ligand binding and EGFR dimerization are essential. EGF binding to EGFR promotes domain rearrangement to expose the dimerization arm in domain II. Since HCP-LCE targets domain II, it was investigated whether the Two-in-One antibody can inhibit EGF-induced EGFR dimerization by measuring the downstream phosphorylation of AKT in EGFR-positive A549 cells. In the presence of HCP-LCE (100 μg/mL), AKT phosphorylation is significantly reduced compared to the EGF-stimulated control (20 ng/mL) (FIG. 4B). Anti-EGFR antibody SEB7 and SEB7×ICI2_H2, which also target EGFR domain II, showed comparable inhibition of AKT phosphorylation as HCP-LCE. The EGFR domain Ill binder Cetuximab completely inhibited EGF-induced phosphorylation of AKT (FIG. 4B) since binding of domain Ill blocked EGF binding. To conclude, EGFR signalling is significantly inhibited by HCP-LCE binding to dimerization domain II without interfering EGF-binding to its receptor.

To investigate HCP-LCE-mediated PD-1/PD-L1 competition, HCP-LCE was loaded onto FAB2G biosensors and was associated to 250 nM PD-L1 pre-incubated with either 250 nM or 1000 nM of PD-1 (PD-1). HCP-LCE exhibited significantly impaired binding to PD-L1 in the presence of PD-1, indicating that the antibody targets and blocks the PD-1/PD-L1 interaction site (FIG. 5A).

Verification of the PD-L1 blockage activity of HCP-LCE was performed in a cell-based context using the Promega® PD-L1 blockade assay kit. HCP-LCE showed notable PD-L1 blockade, although with a less dominant effect compared with ICI2_H2 (FIG. 5B). This diminished EC₅₀ value most probably originates from the lower affinity PD-L1 binding. However, in combination with the original common light chain dFEB4-1, the ICI2 heavy chain exhibited a blockage of the PD-1/PD-L1 interaction comparable to the commercially available antibody durvalumab, as well as a significantly higher affinity PD-L1 binding. SEB7×ICI2_H2 exhibited comparable PD-L1 blockade as HCP-LCE, despite monovalent target binding of the bispecific antibody. The anti-EGFR antibody SEB7 did not interfere with PD-1/PD-L1 interaction even at high concentrations (FIG. 5B). Conclusively, the binding of PD-1 to PD-L1 is significantly inhibited by HCP-LCE, indicating its function as a checkpoint inhibitor.

### Example 7: Cell Titration on A431 Cells

Since HCP-LCE is expected to provide increased tumour-specificity compared to the bispecific antibody SEB7×ICI2_H2 and the corresponding monospecific antibodies due to simultaneous binding of EGFR and PD-L1 at each Fab arm, cell binding experiments were performed on EGFR/PD-L1 double-positive A431 cells by flow cytometry. Cells were stained with the respective antibody at a concentration ranging from 0.12 pM to 500 nM utilizing a four-fold dilution series and binding was verified using an anti-human Fc PE detection antibody. HCP-LCE exhibited cellular binding with an EC₅₀ value of 1.37 nM, while the EC₅₀ value of the bsAb SEB7×ICI2_H2 was about two times higher (FIG. 6). EGFR expression on cancer cells typically strongly exceeds that of PD-L1, as demonstrated by A431 binding of the monospecific antibodies (FIG. 6). The antibodies analysed did not show binding to EGFR/PD-L1 double-negative HEK cells, excluding non-specific cell binding. These data indicate that simultaneous binding of EGFR and PD-L1 at both Fab arms enhances A431 cell binding, compared to monovalent or bivalent target protein binding.

### Example 8: Multispecific Variants

Based on the Two-in-One antibody HCP-LCE (cf. FIG. 7, “HCP-LCE”), five different trispecific variants were designed (cf. FIG. 7, “V1”-“V5”). All variants are based on the LCE as common light chain.
1. A “Knob-into-Hole” (KiH) variant (cf. FIG. 7, “V1”) consisting of HCP-LCE on one side and the heavy chain of the CD16 binder, paired with the HCP-LCE light chain (SEQ ID No.: 17, 18, 19).
2. Two variants with two Fabs (see KiH variant) each side in both possible orientations (cf. FIG. 7, “V2” and “V4”):
   ICI2_outer and NKE14_inner (SEQ ID No.: 20/21)
   NKE14_outer and ICI2_inner (SEQ ID No.: 22/23)
3. both variants were also designed and produced as one-armed (oa) variants (cf. FIG. 7, “V3” and “V5”) (SEQ ID No.: 24, 25, 26, 27).

Purification of antibodies V2 (112_outer) and V4 (NKE14_outer) was performed via Protein A chromatography. For the KiH variant (V1) and the oa variants (V3 and V5) a His-Tag was C-terminally fused to the Hole-Fc and a Twin-Strep-Tag C-terminally cloned to the Knob-Fc. This allowed two-step purification by IMAC, followed by StrepTactin XT chromatography.

The accurate size of the antibodies is in accordance with SOS-PAGE analysis (FIG. 13).

BLI measurements were performed in order to determine the affinity of the trispecific variants to PD-L1, EGFR and 0016. All variants were able to bind the antigens with KOD values shown in the table below.

TABLE 1
BLI-results for different antibody-variants of the present invention

| Variant | Antibody | EGFR | PD-L1 | | CD16 |
|---|---|---|---|---|---|
| V1 | KiH | 570 nM | 104 | nM | 277 nM |
| V2 | ICI2_outer Fab | 460 nM | 97.6 | nM | 382 nM |
| V3 | oaICI2_outer | 763 nM | 115 | nM | 346 nM |
| V4 | NKE14_outer Fab | 603 nM | 125 | nM | 302 nM |
| V5 | oaNKE14_outer | 982 nM | 137 | nM | 342 nM |
| Comparative | NKE14 + LCE | — | — | | 151 nM |
| mAb | | | | | |
| “HCP-LCE” | HCP-LCE | 236 nM | 78.3 | nM | — |
| one arm version | oaHCP-LCE | 294 nM | 79.5 | nM | — |
| of HCP-LCE | | | | | |

To analyse whether all three antigens can be targeted simultaneously, the one-armed variants were loaded onto AHC biosensors and sequentially incubated with the three proteins of interest. Binding to PD-L1 first, EGFR second and CD16 third was considered.

All variants were able to bind the three antigens simultaneously, shown in FIG. 8.

### Example 9: Biophysical Properties of Different Antibodies

TABLE 2
Biophysical properties of HCP-LCE, oaHCP-LCE, LCE, ICI2_H2 and
SEB7xICI2_H2 including affinity, kinetic binding rates, melting temperature and aggregation.

| | K_{D} [nM] | | kₒₙ [M⁻¹ s⁻¹] | | k_{dis} [s⁻¹] | | T_{M} | Aggregation |
|---|---|---|---|---|---|---|---|---|
| Antibody | EGFR | PD-L1 | EGFR | PD-L1 | EGFR | PD-L1 | [° C.] | [%] |
| HCP-LCE | 236 ± | 78.3 ± | 4.15 × | 9.73 × | 9.81 × | 7.62 × | 58.9 | 2.22 |
| | 10.7 | 1.36 | 10⁵ ± | 10⁵ ± | 10⁻² ± | 10⁻² ± | | |
| | | | 1.58 × | 1.37 × | 2.41 × | 7.78 × | | |
| | | | 10⁴ | 10⁴ | 10⁻³ | 10⁻⁴ | | |
| oaHCP- | 295 ± | 117 ± | 3.88 × | 9.29 × | 1.14 × | 1.09 × | 59.6 | 12.44 |
| LCE | 17.1 | 4.15 | 10⁵ ± | 10⁵ ± | 10⁻¹ ± | 10⁻¹ ± | | |
| | | | 1.92 × | 2.62 × | 3.52 × | 2.32 × | | |
| | | | 10⁴ | 10⁴ | 10⁻³ | 10⁻³ | | |
| LCE | — | — | — | — | — | — | 61.1 | 1.27 |
| ICI2_H2 | — | 28.8 ± | — | 1.22 × | — | 3.5 × | 63.0 | 0 |
| | | 0.236 | | 10⁵ ± | | 10⁻³ ± | | |
| | | | | 5.46 × | | 2.40 × | | |
| | | | | 10² | | 10⁻⁵ | | |
| SEB7xICI2_{—} | 7.85 ± | 29.9 ± | 2.34 × | 1.30 × | 1.84 × | 3.90 × | 65.8 | 10.8 |
| H2 | 0.173 | 0.556 | 10⁵ ± | 10⁵ ± | 10⁻³ ± | 10⁻³ ± | | |
| | | | 1.56 × | 1.41 × | 3.87 × | 5.90 × | | |
| | | | 10³ | 10³ | 10⁻⁵ | 10⁻⁵ | | |

### Example 10: CAR-T and/or ADC Applications

Treatment with CAR-T-cells is a cellular immunotherapy strategy that uses the patient's own immune system to fight tumours. T-cells are taken from patients and CARs (chimeric antigen receptor) are introduced into them using viral vectors for tumour cell recognition. An antibody fragment that contains two functionalities in a single molecule (2-in-1) and can simultaneously recognise two tumour antigens that are found in large numbers on the cell surface of solid tumours. The binding to solid tumours would be particularly useful.

The ectodomain of the CAR molecule is a key region responsible for the recognition of target antigens. Several strategies for modification and optimisation have been developed, to design antigen recognition in a way that improves the targeting accuracy of CARs. It has been shown that bispecific CARs that contain two antibody-derived scFv ligand binding domains in tandem and thus simultaneously address two different tumour targets can have an improved efficacy profile (Schneider, Science Transl. Med 2021). Apparently, the increased avidity and specificity of the bispecific CAR improves immunotherapeutic efficacy. This simultaneous targeting also helps to avoid the loss of effect in antigen escape that can occur in heterogeneous solid tumours, as well as to reduce off-target effects. In the case of bispecific tumour cell targeting, it would be desirable to have a binding module that binds both Target A and target B with moderate affinity, but in the AB combination shows a particularly high affinity to tumour cells. The production of such bispecific targeting modules has so far proved difficult. On the one hand, when two scFv molecules are switched in series, it must be ensured that the two scFv domains fold stably and independently of each other and that no light-chain-heavy-chain mixture occurs (so-called chain scrambling), which leads to non-functional molecules. On the other hand, the targeting module should be as small as possible to enable the efficient and productive formation of a cytolytic synapse between the tumour cell and the CAR-T-cell.

To gain access to a bispecific CAR, the VL and VH domains of the Two-in-One antibody are connected through a linker sequence to result in a bifunctional scFv fragment. This fragment is genetically fused to an optional spacer domain, the transmembrane and intracellular domains of an antigen receptor to result in a chimeric antigen receptor. In T-cells, the intracellular signalling domain of the TCR-CD3 complex transduces the necessary signal to kick-start the signalling cascade. Co-stimulatory receptors, especially CD28, are important for sustained signalling, prevention of anergy, and proliferation. In CAR-T-cells, co-stimulatory signals are usually included in-cis with the CD3ζ cytoplasmic domain. The Two-in-One CAR is introduced into T-cells via lentiviral transduction. To this end, CD3+ T-cells, isolated and selected from the Peripheral Blood Mononuclear Cells (PBMC) of healthy donors, are stimulated with anti-CD3/CD28 mAb for T-cell expansion and activation. Cells are transduced with the lentivirus (MOI 5) overnight and transduced cells are expanded. Cell numbers and viability are determined by trypan blue exclusion. To test CAR-T potency, cells are incubated with A431 tumour cells at a tumour cell to CAR-T-cell ratio of 1.25:1 to 10:1 for 72 h. Adherent cells are detached from the plate and stained with anti-CD45 antibodies for T-cell detection and an appropriate antibody for tumour cell detection and analysed by FACS. Alternatively, luciferase-tagged tumour cells are used, and the luciferase activity (as indicator of live target cell) is measured in a luminometer. The relative cell viability is calculated by normalizing relative light unit (RLU) from samples against the RLU from target cells without T-cells.

For the Two-in-One antibody, simultaneous engagement of two different targets by a single molecule may have synergistic or emergent therapeutic effects since for ADC, the cancer-selective delivery of potent cytotoxic payloads may eradicate target-expressing cancer cells while sparing normal healthy tissues.

To generate an ADC, a payload is conjugated to the antibody. Among others, one straightforward strategy is to introduce a recognition sequence for microbial transglutaminase to the C-terminus of the Two-in-One antibody heavy or light chain, respectively such as LLQG. A payload consists of a Val-Cit dipeptide cleavable linker to which the toxin MMAE (monomethyauristatin E) is coupled. The payload contains a triple glycine for enzyme-mediated conjugation. For conjugation, the antibody is mixed with the payload in at least two-fold molar excess and microbial transglutaminase is added and incubated for 6 hours. After protein A purification, the conjugate is tested using A431 cells that simultaneously express both targets, i.e. EGFR and PD-L1. This end, the conjugate is added to the cells at various concentrations ranging from 0 to 100 nM followed by incubation of the conjugate with the tumour cells for 72 h at 37° C., 5% CO₂. Cell viability is determined using the celltiter 96® aqueous one solution cell proliferation assay (Promega) by recoding absorbance at 490 nm in 96 well plate reader and the IC50 is determined on the basis that the absorbance of the formed formazan is directly proportional to the number of living cells in the culture.

### Example 11: Trispecific Variants Based on the Two-In-One Antibody HCP-LCE

Based on the Two-in-One antibody HCP-LCE, three different trispecific variants were designed. For this, a new CD16 binder was found, which carries the light chain LCE. All variants are based on the LCE as common light chain.

A Knob-into-Hole (KiH) variant consisting of HCP-LCE on one side and the new CD16 binder (HC16) on the other side, paired with the HCP-LCE light chain.

Two variants with two Fabs (see KiH variant) each side in both possible orientations:
ICI2_outer/HC16_inner (Seq ID Nos.: 30/31)
HC16_outer/ICI2_inner (Seq ID Nos.: 28/29)
   both variants were also designed and produced as one-armed (oa) variants.

The purification of antibodies ICI2_outer/HC16_inner and HC16_outer/ICI2_inner was performed via Protein A chromatography. For the HC16/ICI2 KiH variant and the oaHC16_outer variant a His-Tag was C-terminally fused to the Hole-Fc and a Twin-Strep-Tag C-terminally cloned to the Knob-Fc. This allowed two-step purification by IMAC, followed by StrepTactin XT chromatography.

The accurate size of the antibodies was in accordance with reducing and non-reducing SDS-PAGE analysis.

BLI measurements were performed in order to determine the affinity of the trispecific variants to PD-L1, EGFR and CD16. All variants were able to bind the antigens with K_{D} values shown in Table 3 below.

TABLE 3
K_{D} values of all variants

| Antibody | EGFR | PD-L1 | | CD16 | |
|---|---|---|---|---|---|
| KiH | 320 nM | 57.7 | nM | 21.9 | nM |
| ICI2_outer | 416 nM | 56.7 | nM | 24.6 | nM |
| oaICI2_outer | 257 nM | 51.8 | nM | 25.6 | nM |
| HC16_outer | 331 nM | 68 | nM | 17 | nM |
| oaHC16_outer | 463 nM | 64.8 | nM | 19.9 | nM |

To analyse whether all three antigens can be targeted simultaneously, the one-armed variants/KiH variant were loaded onto AHC biosensors and sequentially incubated with the three proteins of interest. Binding to PD-L1 first, EGFR second and CD16 third was considered. All variants were able to bind the three antigens simultaneously.

In the EGF competition assay it turned out that the antibodies do not interfere binding of EGF to EGFR, but in the PD-1 competition assay it was shown that the antibodies inhibit binding of PD-1 to PD-L1.

EC₅₀-values of the antibodies were measured as explained before. Table 4 shows the results:

TABLE 4
EC₅₀- values of all variants

| | EC₅₀ |
|---|---|
| HC16_outer | 0.6103 |
| ICI2_outer | 2.098 |
| HC16/ICI2 KiH | 3.926 |
| oaHC16_outer | 6.595 |
| 2in1 | 0.2452 |

To investigate immune cell stimulation and efficiency of cell killing, the Promega ADCC luminescent reporter assay was used. As target cells, EGFR/PD-L1 double positive A431 cells were used. The 2in1 antibody containing a LALA mutation failed to mediate ADCC effects as expected (cf. FIG. 20).

### Example 12: Tetrafunctional T-Cell Engaging Antibody with Risk Mitigation of Cytokine Release Syndrome

### 12.1 Cell Culture

A431 (ACC 91), A549 (ACC 107), and THP-1 (ACC 16) cells were obtained from DSMZ (German Collection of Microorganisms and Cell Cultures GmbH). Adherent cell lines (A431, A549) were maintained in Dulbecco's Minimal Eagle Medium (DMEM) supplemented with 10% FBS and 1× penicillin/streptomycin. THP-1 cells were maintained in RPMI 1640 supplemented with 10% FBS and 1× penicillin/streptomycin. Cells were subcultured every 2-3 days and incubated at 37° C., 5% CO2.

### 12.2 Cloning, Production and Purification of Antibody Constructs

A trispecific T-cell engager with TME and cytokine release modulation (TriTECM) was generated.

The HCP-LCE IgG backbone was used as a starting block. For the different scFvs, gene fragments were ordered from Twist Biosciences based on their published protein sequences with respective SapI overhangs. The various architectures were cloned using Q5 DNA Polymerase and subsequent SapI-mediated Golden Gate Cloning (GGC). The sequences were verified by Sanger Sequencing at Microsynth (Göttingen). An effector-silenced Fc-backbone carrying LALA mutations was used, as well as the Knob-into-Hole (KiH) technology to ensure heterodimerisation.

For the production of the antibodies, Expi293-F (Thermo Fisher Scientific) cells were transfected with Expifectamine293 (Thermo Fisher Scientific), following the manufacturer's instructions. The cells were cultured in Expi293 Expression Medium (Thermo Fisher Scientific), sub-cultured every 3-4 days and incubated at 37° C., 8% CO2. Transfections were harvested 5 days post-transfection. The supernatants were sterile filtered and supplemented with 18.1 mL/L BioLock (IBA Lifesciences). Purification of all KiH variants was performed by a two-step purification using an AKTA Pure25 (Cytiva Lifesciences); a His-tag purification using a HisTrap™ excel column (Cytiva Lifesciences) was performed followed by TwinStrep®-tag purification using a Streptactin®XT 4Flow® column. In this manner, heterodimeric heavy chains were ensured.

Due to its ability to bind and block both EGFR and PD-L1 pathways and having two binding sites for each target, the inventive two-in-one antibody was chosen as the backbone, carrying an effector-silenced Fc to minimise cytotoxicity and extend the half-life. This molecule carries dual targeting abilities in a generic IgG1 antibody format, where the light chain mainly contributes to EGFR-binding and the heavy chain to PD-L1-binding. Moreover, while individual binding to each target occurs with moderate affinity, tumour cells expressing both antigens were bound with high affinity, indicating that the antibody, besides being able to inhibit the PD-1/PD-L1 axis, displays enhanced tumour selectivity.

For CD3ε-engagement, a fully human monoclonal antibody (CD3ε-engager) was considered, from which a single-chain variable fragment (scFv) was derived.

CD3-binding and IL-6R-modulating scFvs could be derived from all existing anti-CD3 and anti-IL-6R/IL-6 antibodies, such as muromonab, otelixizumab, teplizumab, visilizumab, foralumab, tocilizumab, sarilumab, siltuximab, sirukumab, olokizumab, satralizumab, clazakizumab.

In the proof-of-concept study described here, the CD3ε-engager was used as a CD3-based T-cell engager.

For inhibition of the IL6-IL-6R-gp130 pathway, a scFv was generated (anti-IL-6R scFv) which exhibits exceptional affinity to the IL-6Ra with 61.9 pM, approximately 20-fold higher than tocilizumab, and is able to block both the classical- and trans-mediated signalling pathways. Furthermore, recent studies showed the ability of the anti-IL-6R scFv to modulate CRS events in SARS-CoV2-infected patients.

With these building blocks, different TCEs were designed. To have better control over the positioning of the scFvs added, all molecules were designed with the Knob into Hole (KiH) technology. To avoid hyper-cytokine release, a single CD3-binding scFv was fused onto the HCP-LCE KiH backbone. The distance and relative positioning of TAA and CD3 binding modules of T-cell recruiting antibodies matter, influences their ability to bridge the immune synapse between a tumour-associated antigen and the TCR on the surface of T cells. For this purpose, a classical approach was taken, fusing the anti-CD3E scFv to the N-terminus of the VH domain with a (G₄S)₃ linker, providing a relatively small distance between CD3-engagement and the surface of the tumour cell upon binding. Conversely, a C-terminal fusion to the Knob-Fc with a (G₄S)₃ linker was also generated, providing the largest distance between the tumour-binding Fab and the engaging T cell (FIG. 14A). The anti-IL-6R scFv was placed either at the C-termini of both heavy chain (HC) Fc-fragments or only at the Hole-Fc heavy chain, respectively. The control variants are all depicted and named in FIG. 14B. For simplicity, the nomenclature is based on the positioning of the scFvs (either N- or C-terminal) to the heavy chain and either for the Knob (K) or Hole (H) HCs. As an example, a fusion of anti-CD3ε scFv N-terminal to the VH of the Knob HC together with a C-terminal fusion of the anti-IL-6R scFv to both the Knob and Hole HC is denoted as (αCD3εK(αIL-6R)+H(αIL-6R). The parental two-in-one antibody is termed K+H.

### 12.3 Biophysical Characterization (SDS-PAGE, Thermal Stability)

Production of the antibodies was performed in Expi293-F cells by transient transfection with expression plasmids using Expifectamine293. To ensure exclusive isolation of heterodimers, a Twin-StrepII-tag sequence was placed at the C-terminus of the CH3 domain of the Knob-Fc, while a His6-tag was placed C-terminally to the Hole-Fc, as previously described.

Subsequently, the cell culture supernatant was purified via a two-step purification, namely an IMAC followed by StrepTactin purification to purify only heterodimeric antibodies carrying both a Knob- and a Hole-HC.

To characterise the produced antibodies, SDS-PAGE analysis was performed. To this end, 4 μg purified mAb were loaded onto a Mini-PROTEAN TGX 4-15% Gel (BioRad) with either reducing- or non-reducing-Lämmli buffer, and subsequently stained with Coomassie.

SDS-PAGE analysis revealed all the expected heavy- and light-chain bands under reducing conditions, as well as the expected molecular size under non-reducing conditions with no degradation products (FIG. 14C). The antibodies unveiled appropriate yields for all variants, with the yield decreasing with increasing number of fused scFvs as expected (Table 5).

For thermal stability determination, antibodies were incubated with SYPRO Orange (Thermo Fisher Scientific) and a thermal shift assay was performed using a CFX Connect Real-Time PCR System (BioRad). The temperature gradient was set from 10° C. to 95° C. with an increment of 0.5° C./10 s. The derivatives of the melt curves were determined with the CFX Maestro software to determine the melt temperature (TM).

Thermal stability investigated by SYPRO Orange revealed melting temperatures between 63.50° C. and 78.00° C., with the parental K+H exhibiting 66.50° C., revealing no large reduction in thermal stability (Table 5).

TABLE 5
Yields and thermal stability of all variants.

| Variants | Yield per 1 L (mg) | Thermal stability (° C.) |
|---|---|---|
| K + H | 122.344 | 66.50 |
| K + H(αIL-6R) | 54.512 | 70.00 |
| K(αCD3ε) + H | 63.170 | 78.00 |
| K(αCD3ε) + H(αIL-6R) | 22.478 | 69.50 |
| K(αCD3ε) + H(αIL-6R) | 31.635 | 63.50 |
| (αCD3ε)K + H | 101.765 | 74.50 |
| (αCD3ε)K(αIL-6R + | 23.643 | 64.00 |
| H(αIL-6R) | | |
| oa(αCD3ε)K(αIL-6R) | 26.074 | 71.00 |

Affinity towards the different antigens was determined via biolayer interferometry (BLI). Antibodies were loaded onto AHC biosensors and varying concentrations of antigen were associated to the mAb. The determined affinities were in similar ranges to those cited in literature from previous studies, with only minor changes where the fused scFvs could presumably interfere with binding (Table 6). Nevertheless, the affinity to neither EGFR nor PD-L1 was greatly influenced by N- or C-terminal scFv fusions. The affinity towards soluble IL-6R (sIL-6R-TS) was not determined for all IL-6R-binding variants due to the very slow dissociation, however both K(αCD3ε)+H(αIL-6R) and (αCD3ε)K(αIL-6R)+H(αIL-6R) showed single-digit nanomolar affinity towards IL-6R.

TABLE 6
Affinities towards soluble, monomeric EGFR,
PD-L1 or IL-6R as determined by BLI.

| | K_{D} (nM) | | |
|---|---|---|---|
| Variant | His₆-EGFR-TS | His₆-PD-L1-TS | sIL-6R-TS |
| K + H | 173 ± 10.8 | 92.9 ± 2.35 | — |
| K + H(αIL-6R) | 355 ± 19.4 | 68.8 ± 2.02 | < 0.001* |
| K(αIL-6R) + | 503 ± 20.0 | 70.7 ± 2.51 | < 0.001* |
| H(αIL-6R) | | | |
| K(αCD3ε) + H | 228 ± 12.4 | 105.0 ± 2.80 | — |
| K(αCD3ε) + | 172 ± 10.5 | 112 ± 3.14 | 1.80 ± 0.188 |
| H(αIL-6R) | | | |
| (αCD3ε)K + H | 236 ± 11.5 | 108.0 ± 2.85 | — |
| (αCD3ε)K(αIL-6R) + | 271 ± 14.4 | 130 ± 3.73 | 2.71 ± 0.0848 |
| H(αIL-6R) | | | |

*curves were not fittable due to very slow dissociation that precludes exact off-rate determination.
TS—TwinStrepII tag.

### 12.4 Affinity Determination, Simultaneous Binding and Competition Assays Via BLI

Affinities were determined using biolayer interferometry (BLI) on an Octet RED96. For kinetics determination, 60 nM antibody was loaded onto anti-human Fc capture (AHC) biosensors and incubated with varying concentrations of antigen, in a range from 0-500 nM for EGFR and PD-L1, and 0-200 nM for IL-6R. Fitting of the curves for affinity determination was performed based on Savitzky-Golay filtering and a 1:1 Langmuir binding model.

For simultaneous binding studies, 10 μg/ml antibody was loaded onto an AHC biosensor and sequentially associated to 200 nM IL-6R, followed by 500 nM EGFR and PD-L1. A negative control with PBS was included to eliminate unspecific binding of the self-produced antigens.

Competition assays for PD-L1/PD-1 and IL-6/IL-6R were performed as previously described.

To ensure the tumour-targeting ability of the two-in-one backbone remained intact, A431 cells were examined that are positive for both EGFR and PD-L1, revealing very similar on-cell affinities for all variants (Table 7). The parental K+H antibody showed an affinity of 2.5 nM, similar to previous studies, and the two TriTECM molecules revealed 12.3 and 21.2 nM for (αCD3ε)K(αIL-6R)+H(αIL-6R) and K(αCD3ε)+H(αIL-6R), respectively. Nonetheless, very good on-cell affinities were measured for all variants. CD3 binding was observed on Jurkat cells, with the (αCD3ε)K+H and K(αCD3ε+H variants binding with two-digit nanomolar affinities. A reduction in CD3-binding was observed for both TriTECM variants, presumably due to different conformations of the molecules (Table 7). Lastly, IL-6R-binding was confirmed on macrophage-like THP-1 cells showing similar binding profiles for the variants containing a anti-IL-6R scFv (Table 7).

TABLE 7
On-cell Affinities on A431 (EGFR⁺/PD-L1⁺), Jurkat (CD3⁺) and THP-1
(IL-6R⁺/PD-L1⁺) as determined by flow cytometry.

On-cell affinity (nM)

| Variant | A431 | Jurkat | THP-1 |
|---|---|---|---|
| K + H | 2.50 | — | — |
| K + H(αIL-6R) | 4.30 | — | 3.10 |
| K(αCD3ε) + H | 4.50 | 195.90 | — |
| K(αCD3ε) + H(αIL-6R) | 21.24 | 213.80 | 36.66 |
| (αCD3ε)K + H | 1.82 | 25.38 | — |
| (αCD3ε)K(αIL-6R) + H(αIL-6R) | 12.34 | n.d. | 10.08 |

“—” represents no significant binding, “n.d.” represents non-determinable affinities.

For T-cell engagers, simultaneous binding and engagement of both tumour- and T-cells is crucial. Accordingly, simultaneous binding of Calcein AM-stained A431 tumour cells and pre-incubated CD3-stained Jurkat cells was investigated by flow cytometry (FIG. 15A).

Quantification of FITC+/PE+ and FITC−/PE+ binding events were considered as successful co-binding, as both CD3-binding on Jurkat cells was observed through the antibodies, as well as to A431 cells via EGFR and PD-L1 (FIG. 15B). The two best candidates were (αCD3ε)K+H and (αCD3ε)K(αIL-6R)+H(αIL-6R), followed by K(αCD3ε)+H and K(αCD3ε)+H(αIL-6R). Weaker simultaneous binding was observed for (αCD3ε)K(αIL-6R)+H(αIL-6R) and K(αCD3ε)+H(αIL-6R) compared to their counterparts without anti-IL-6R scFv, as expected from their reduced on-cell affinity, however still significant compared to K+H or K+H(αIL-6R) which are not able to bind Jurkat cells due to the absence of the CD3 binding module.

Furthermore, simultaneous binding to IL-6R, EGFR and PD-L1 was confirmed via BLI by loading the different constructs onto AHC biosensors and associating to the three antigens sequentially for 300 seconds each (FIG. 16). A one-armed variant of (αCD3ε)K(αIL-6R+H(αIL-6R) (dubbed oa(αCD3ε)K(αIL-6R)) was generated to test whether a single arm could still bind all three targets. Interestingly, the one-armed variant was also able to bind IL-6R, EGFR and PD-L1, though with decreased layer thickness increase due to only monovalent binding to each antigen. This indicated that the two-in-one Fab retained its ability to simultaneously bind EGFR and PD-L1 with scFvs fused to its N-terminus. Overall, the tetraspecificity of both TriTECM molecules was confirmed.

### 12.5 Ligand Competition and Signaling Pathway Inhibition

For a full therapeutic effect, it is important that the antibodies binding to EGFR, PD-L1 and IL-6R have antagonistic effects. Sarilumab is well-known to compete with the IL-6 binding site on the IL-6R, and the previously identified parental K+H antibody competes with PD-1 for binding to PD-L1. Via BLI, a competition assay was performed for both TriTECM molecules and sarilumab or K+H as controls. For IL-6/IL-6R competition, the (αCD3ε)K(αIL-6R)+H(αIL-6R) construct inhibited equally well compared with sarilumab IL-6R binding and ligand competition, however, the K(αCD3ε)+H(αIL-6R) showed weaker competition (FIG. 17A). This is presumably due to monovalent IL-6R binding, whereas both sarilumab and (αCD3ε)K(αIL-6R+H(αIL-6R) are bivalent. For PD-L1, all antibodies displayed good binding to PD-L1, which became weaker upon pre-incubation with PD-1, confirming competition of binding (FIG. 17B).

As the parental K+H antibody binds to domain II of EGFR, it does not compete with its ligand EGF. However, it still inhibits the EGF-mediated signaling pathway. Similarly, sarilumab is also able to inhibit IL-6-mediated signaling. Hence, pAKT and pSTAT3 were measured for EGF- and IL-6-mediated signaling inhibition, respectively. For IL-6/IL-6R, pSTAT3 analysis by HTRF assays revealed dose-dependent inhibition for both TriTECMs and the K(αIL-6R)+H(αIL-6R) control mAb (FIG. 17). While a slight reduction was noticed with 300 nM of either (αCD3ε)K+H or K(αCD3ε)+H, the inhibition was not as high as for the molecules with anti-IL-6R scFv and similar to that of K+H. For EGF/EGFR signaling, pAKT was measured and all antibodies exhibited dose-dependent inhibition compared to the EGF control, all very similar to the parental K+H antibody (FIG. 17D). Furthermore, to investigate whether the antibodies could inhibit both pathways simultaneously, cells were pre-incubated with mAbs and then stimulated with a combination of EGF and IL-6. HTRF analysis revealed dose-dependent inhibition of both pathways as seen by pSTAT3 and pAKT inhibition (FIG. 17E, F). Thus, despite minor differences in EGFR/PD-L1 binding of the TriTECMs as measured by affinity determination or on-cell affinities, their functionalities were retained compared to the parental two-in-one antibody in the K+H format.

### 12.6 T-Cell Activation Assays

A fundamental aspect of a T-cell engager is the ability of the CD3-binding moiety to activate T cells upon binding and engagement with tumor cells. To this end, a commercially available kit was used to measure activation of the NFAT pathway after engagement of effector (TCR/CD3 cells) and tumor cells (A431).

To determine the potency of T-cell engagement, the T-cell activation Bioassay (NFAT) (Promega) was performed following the manufacturer's instructions. In brief, 4×104 A431 cells/well were seeded in sterile 96-well plates and allowed to adhere overnight. The following morning, 1×105 effector cells/well were added with the indicated antibody dilutions (3× concentrations). The co-culture was incubated for 6 h at 37° C., 5% CO₂, followed by the addition of Bio-Glo reagent. After 5-10 min, the plate was measured on a luminescence plate reader.

Potent T-cell activation was observed for the two variants containing only an additional anti-CD3ε scFv with EC50 of 0.59 and 0.63 nM for (αCD3ε)K+H and K(αCD3ε)+H, respectively (FIG. 18A,B). For the TriTECMs, a 5- and 10-fold reduction in EC50 was observed compared to their counterparts lacking sarilumab, with 2.51 and 5.60 nM for (αCD3ε)K(αIL-6R)+H(αIL-6R) and K(αCD3ε)+H(αIL-6R), respectively (FIG. 18A,B). Nonetheless, significant T-cell activation was observed, meaning no complete loss-of-function occurred through the diminished CD3 binding on effector cells (Table 6).

While immortalized cell lines can provide an estimate of in vitro potency, human peripheral blood mononuclear cells (PBMCs) isolated from healthy donors are a more reliable source.

Subsequently, PBMCs were incubated with 30 nM of the indicated antibodies for 48 h and the early and late-stage T-cell activation markers CD69 and CD25, respectively, were measured by flow cytometry (FIG. 18C). While the control antibodies and blank measurements all showed similar levels, a significant increase in both activation markers (upper right quadrant) was observed where the CD3-binding scFv was present, as expected. Quantification of the different quadrants showed increased expression in CD25/CD69 and CD25 for all four anti-CD3ε scFv-containing molecules, with a significant reduction for (αCD3ε)K(αIL-6R)+H(αIL-6R) compared to (αCD3ε)K+H, as previously observed (FIG. 17E, F). Not many CD69+/CD25−events were observed compared to the controls, however this could be due to the gradual decrease of CD69 with longer incubation times (FIG. 18D).

In summary, T-cell activation was observed for all molecules containing a CD3-binding scFv, with the (αCD3ε)K(αIL-6R)+H(αIL-6R) showing the most attenuated T-cell activation, which might aid in the search for T-cell engagers with increased safety profiles.

12.7 Attenuated Cytokine Release and Cytotoxicity with TriTECMs

One of the large drawbacks of T-cell engagers is the increase in pro-inflammatory cytokines such as TNFα and IFNγ upon T-cell activation, leading to the hyperactivation of macrophages. A hyperactivation of macrophages leads to the secretion of high levels of IL-6 and other cytokines, ultimately resulting in a cytokine storm. To investigate whether the addition of the anti-IL-6R scFv module resulted in decreased pro-inflammatory cytokine release, PBMCs were stimulated with 30 nM of the respective antibodies and stimulated over 72 h. Thereafter, the release of pro-inflammatory IFNγ was measured in the supernatant (FIG. 19A), and the cells themselves were stained with an APC-conjugated anti-CD25 antibody to measure T-cell activation (FIG. 19B).

In both cases, significant increases in either IFNγ or CD25 were observed compared to the controls (K+H, K+H(αIL-6R), and K(αIL-6R)+H(αIL-6R)). In the case of IFNγ, a decrease was noted with the TriTECM (αCD3ε)K(αIL-6R)+H(αIL-6R) compared to (αCD3ε)K+H, hinting at both an attenuated activation and a decrease in cytokine release due to the anti-IL-6R scFv (FIG. 19A). This effect was also noted for K(αCD3ε)+H(αIL-6R) compared to K(αCD3ε)+H but to a smaller extent, presumably due to monovalent IL-6R binding (FIG. 19A). CD25 staining revealed significant T-cell activation with the TriTECM molecules compared to their controls (FIG. 19B).

To ensure the attenuation of CD3-binding still led to decent T-cell-mediated cytotoxicity, cell killing of tumor cells by co-incubation with PBMCs was investigated. Dose-dependent cytotoxicity was observed through PI staining after 24 h co-incubation (FIG. 19C, D), with a slight reduction of activation for both TriTECM variants compared to their controls without anti-IL-6R-binding moieties. Longer incubation times of 48 h revealed T-cell-mediated cytotoxicity as measured by lactate dehydrogenase (LDH) release (FIG. 19E). While the tetraspecific molecules resulted in lowered cytotoxicity, these results, combined with previous T-cell activation studies (FIG. 18) prove the attenuated nature of TriTECMs and their potential use as TCEs with a cytokine release and activity modulating function.

### 12.7.1 PBMC Isolation

Peripheral blood mononuclear cells (PBMCs) were obtained from buffy coats from healthy human donors obtained from the Deutsche Rotes Kreuz (Frankfurt). To this end, 25 ml blood was mixed 1:1 with PBS− 2% FBS (PBS-F) and PBMCs were purified using SepMate-50 tubes following the manufacturer's instructions (StemCell Technologies). The isolated PBMCs were frozen in 80% RPMI 1640, 20% FBS and 10% DMSO and thawed directly when required. All work was performed according to local ethics and welfare regulations.

### 12.7.2 Cytokine Release Assay

For cytokine release assays, PBMCs were thawed and seeded onto 48-well plates at 3×105 cells/well. The desired antibody concentration was added to the cells, and the mixture was incubated at 37° C., 5% CO₂ for 72 h. After 72 h, the supernatant was collected and centrifuged to remove any cell debris. The cells were separated for further flow cytometric analysis. A minimum of four healthy donors were used and at least biological duplicates were measured.

### 12.7.3 Signaling Assays

To detect phosphoproteins and signalling inhibition, HTRF (Perkin Elmer) kits were used. Therefore, 5×104 A549 cells/well were seeded onto 96-well tissue-culture plates and allowed to adhere for 4 h, cells were subsequently serum-starved overnight. The serum-starved cells were pre-treated with the desired antibody concentrations for 1 h before the addition of 20 ng/ml ligand for 10 or 40 min for EGF and IL-6, respectively. Immediately afterwards, the cells were washed with ice-cold PBS and lysed in the appropriate lysis buffer according to the manufacturer's instructions. The investigation of the EGF/EGFR pathway was performed using the Phospho-Akt (Ser4739 cellular HTRF Kit (Perkin Elmer), and the IL-6/IL-6R pathway was investigated using the Phospho-STAT3 (Tyr705) cellular HTRF kit (Perkin Elmer). Data was graphed using GraphPad Prism 8.0.1.

### 12.7.4 Cytokine ELISAs

Quantification of secreted cytokines from cell culture supernatants was measured using commercially available ELISAs following the manufacturer's instructions. ELISA kit was purchased from R&D Systems: Human IFN-gamma DuoSet ELISA (DY285B).

### 12.7.5 T-Cell-Mediated Cytotoxicity

Evaluation of cytotoxicity was performed by incubating A431 cells with PBMCs (effector-to-target ratio 10:1) in 96-well tissue culture plates with the desired antibody concentration for 24-48 h. After different time points, the supernatant was used to measure released LDH by the LDH-Glo Cytotoxicity Assay (Promega) following the manufacturer's instructions. Plates were observed under a bright-field microscope to evaluate potential cell killing. Further, dead cell staining was performed by trypsinizing and collecting all cells, staining with propidium iodide (PI) (Sigma Aldrich) and measuring PE fluorescence using a flow cytometer.

12.7.6 Flow cytometry

Flow cytometric analysis was performed by first washing the cells of interest with ice-cold PBS-2% FBS (PBS-F). Cells were then stained with primary antibodies and incubated on ice for 30 min. Subsequently, the cell pellets were washed with ice-cold PBS-F and resuspended in an appropriate volume of PBS-F for measurement using a CytoFlex S (Beckman Coulter) flow cytometer. If the primary antibody was not directly conjugated, a secondary antibody incubation was performed on ice for 15 min before measurement. The antibodies used in this study were: anti-human Fc-PE (Thermo Fisher Scientific), anti-human CD69-FITC (Thermo Fisher Scientific), anti-human CD25-APC (Thermo Fisher Scientific). Dot plots were generated using FlowJo V10 software.

1. A monoclonal antibody or antigen-binding fragment, or a substantially similar variant thereof characterized in that at least one Fab-region binds specifically to epidermal growth factor receptor (EGFR) and programmed cell death-ligand 1 (PD-L1).

2. The antibody or antigen-binding fragment or substantially similar variant thereof according to claim 1, comprising at least one Fv-region binding specifically to epidermal growth factor receptor (EGFR) and programmed cell death-ligand 1 (PD-L1).

3. The antibody or antigen-binding fragment or substantially similar variant thereof according to claim 1, wherein the paratopes, on said antibody or antigen-binding fragment or substantially similar variant thereof do not overlap.

4. The antibody or antigen-binding fragment or substantially similar variant thereof according to claim 1, capable of inhibiting EGFR-signalling and blocking the PD-1/PD-L1 interaction.

5. The antibody or antigen-binding fragment or substantially similar variant thereof according to claim 1, wherein the EGFR binding is at dimerization domain II of EGFR and wherein the antibody prevents binding of PD-L1 to PD-1 and/or competes for binding to PD-L1 with durvalumab.

6. The antibody or antigen-binding fragment or substantially similar variant thereof according to claim 1, comprising
a heavy chain CDR1 (H-CDR1) domain with the amino acid sequence of SEQ ID No.: 03; and/or
a heavy chain CDR2 (H-CDR2) domain with the amino acid sequence SEQ ID No.: 04; and/or
a heavy chain CDR3 (H-CDR3) domain with the amino acid sequence of SEQ ID No.: 05; and/or
and/or
a light chain CDR1 (L-CDR1) domain with the amino acid sequence of SEQ ID No.: 06; and/or
a light chain CDR2 (L-CDR2) domain with the amino acid sequence of SEQ ID No.: 07; and/or
a light chain CDR3 (L-CDR3) domain with the amino acid sequence of SEQ ID No.: 08.

7. The antibody or antigen-binding fragment or substantially similar variant thereof according to claim 1, comprising
at least 95% sequence identity to a heavy chain V_{H}-region with the amino acid sequence selected of SEQ ID No.: 01;
and/or
at least 95% sequence identity to a light chain V_{L}-region domain with the amino acid sequence of SEQ ID No.: 02.

8. The antibody or antigen-binding fragment or substantially similar variant thereof according to claim 1, comprising a first amino acid sequence identical to the amino acid sequence of SEQ ID No.: 01 and a second amino acid sequence identical to the amino acid sequence of SEQ ID No.: 02.

9. The antibody or antigen-binding fragment or substantially similar variant thereof according to claim 1, comprising a heavy chain variable region of the amino acid sequence in SEQ ID No.: 01 and a light chain variable region of the amino acid sequence in SEQ ID No.: 02.

10. The antibody or antigen-binding fragment or substantially similar variant thereof according to claim 1, characterized by one or more of the following:
human or humanized;
also capable of binding to at least a further target protein selected from the group consisting of Fcγ-receptor IIIa/IIIb (CD16a/b), cluster of differentiation 155 (CD155), MHC class I chain-related proteins A and B (MicA/MicB), cluster of differentiation 159 (CD159), a NKG2 receptor for natural killer cells (NK cells), cluster of differentiation 276 (CD276), hepatitis A virus cellular receptor 2 (HAVCR2), T-cell immuno-receptor with Ig and ITIM domains (TIGIT), B-cell maturation antigen (BCMA or BCM), interleukin-3 receptor (CD123), natural cytotoxicity triggering receptor 3 (NKp30), natural cytotoxicity triggering receptor 3 (NKp44), natural cytotoxicity triggering receptor 3 (NKp46), cluster of differentiation 27 (CD27), cluster of differentiation 96 (CD96), NKRP1A, cluster of differentiation 355 (CD355), and lymphocyte function-associated antigen 1 (LFA-1), and/or any combination thereof,
capable of binding specifically to PD-L1-protein with an affinity (KD) of between 50 and 500 nM;
capable of binding specifically to EGFR-protein with an affinity (KD) of between 150 and 1000 nM;
capable to specifically bind to a tumour cell, wherein the tumour cell is characterized by being double-positive for PD-L1 and EGFR;
inhibits the dimerization of EGFR, but not the binding of EGF to EGFR;
inhibits EGFR-pathway signalling at least 2-fold or more;
inhibits the binding of PD-L1 to PD-1 with an EC₅₀ of between 5 and 500 nM;
binds to double-positive cells with EGFR and PD-L1 with an EC₅₀ of between 0.5 and 20 nM;
being further modified to an antibody-drug conjugate (ADC) by being conjugated to a therapeutic agent;
being further modified to extend its half-life by albumin-fusion, polyaminoacid fusion, fc fusion, or a polymer-conjugation with polyethylene glycol, sialic acid, starch and/or hyaluronic acid.

11. An isolated nucleic acid molecule encoding the monoclonal antibody or antigen-binding fragment, or substantially similar variant thereof according to claim.

12. An expression vector comprising the nucleic acid molecule according to claim 11.

13. A method of producing the antibody or antigen-binding fragment or substantially similar variant thereof according to claim 1
i. Selecting a first and a second human target protein;
ii. Creating a first yeast surface display (YSD) library, comprising the following steps:
a. Immunize an animal of avian species, with the first target protein;
b. Isolate variable heavy chain (VH) genes from cDNA derived from said immunized animal of avian species;
c. Insert the VH genes into a vector designed for expression, secretion, and display of proteins on the extracellular surface of a first population of Saccharomyces cerevisiae (yeast) cells, and transform the first population of yeast cells with said vector;
d. Insert genes for a common variable light chain (VL) (SEQ ID No.: 16) into an expression vector designed for expression, secretion, and display of proteins on the extracellular surface of a second population of Saccharomyces cerevisiae cells, and transform the second population of yeast cells with said vector;
e. Combine the two yeast cell populations by yeast cell mating, resulting in diploid yeast cells displaying VH domains from the PD-L1-immunized animal of avian species, paired with the common variable light chain (VL) (SEQ ID No.: 16);
f. Screen by fluorescence activated cell sorting (FACS) for the binding to the first target protein and isolate yeast cells displaying antibodies which specifically bind to the first target protein;
iii. Creating a second yeast surface display (YSD) library, comprising the following steps:
a. Immunize an animal of avian species, preferably a chicken, with the second target protein; preferably EGFR if the first antigen was PD-L1, or PD-L1 if the first antigen was EGFR;
b. Isolate variable light chain (VL) genes from cDNA derived from said immunized animal of avian species;
c. Insert genes for the common variable heavy chain (V_{H}) gene (SEQ ID No.: 15) into a vector designed for expression, secretion, and display of proteins on the extracellular surface of a third population of Saccharomyces cerevisiae cells, and transform the third population of yeast cells with said vector;
iv. Combining the yeast cells from the first YSD-library carrying heavy chains (VH) specifically binding to the first target protein with the second YSD-library carrying the light chains (VL) from the animal of avian species immunized with the second target protein, by yeast mating
v. Screening the resulting yeast cells by fluorescence activated cell sorting (FACS) for binding to both target proteins; and/or inhibition of each of the two target proteins;
vi. Identifying the yeast cells comprising antibodies comprising heavy chains specifically binding to the first target protein and comprising light chains specifically binding to the second target protein;
vii. Isolating a bispecific, tetravalent humanized or a human monoclonal antibody or antigen-binding fragment binding to both target proteins,
viii. Receiving the antibody or antigen-binding fragment or a substantially similar variant thereof according to claim 1.

14. A pharmaceutical composition comprising the bispecific monoclonal antibody or antigen-binding fragment, or substantially similar variant thereof according to claim 1, and a pharmaceutically acceptable carrier.

15. A bispecific monoclonal antibody or antigen-binding fragment, or substantially similar variant thereof according to claim 1 for the use in the identification, prevention and/or treatment of a disease or condition in a patient.


SEQUENCE LISTING (41 sequences)

SEQ ID NO: 1 - protein, 125 aa, Gallus sp.
  REGION 1..125: note = HCP-LCE VH-Sequence
AVTLDESGGGLQTPGGALSLVCKASGFDFSSYDMAWVRQAPGKGLEWVAGIKSNGPWTNYGAAVKGRATISRDNGQSTVRLQLNNLRAEDTAIYYCAKPGAGYVGWDAAGTIDSWGHGTEVIVSS

SEQ ID NO: 2 - protein, 106 aa, Gallus sp.
  REGION 1..106: note = HCP-LCE VL-Sequence
ALTQPSSVSANLGETVKITCSGGAYSDCGWYQQKSPGSAPVTVIYYNDKRPSDIPSRFSGSTFGSTATLTITGVQAEDEAVYYCGGYDSRTGYVGVFGAGTTLTVL

SEQ ID NO: 3 - protein, 5 aa, Gallus sp.
  REGION 1..5: note = HCP-LCE CDR-H1
SYDMA

SEQ ID NO: 4 - protein, 17 aa, Gallus sp.
  REGION 1..17: note = HCP-LCE CDR-H2
GIKSNGPWTNYGAAVKG

SEQ ID NO: 5 - protein, 16 aa, Gallus sp.
  REGION 1..16: note = HCP-LCE CDR-H3
PGAGYVGWDAAGTIDS

SEQ ID NO: 6 - protein, 9 aa, Gallus sp.
  REGION 1..9: note = HCP-LCE CDR-L1
SGGAYSDCG

SEQ ID NO: 7 - protein, 8 aa, Gallus sp.
  REGION 1..8: note = HCP-LCE CDR-L2
YNDKRPSD

SEQ ID NO: 8 - protein, 12 aa, Gallus sp.
  REGION 1..12: note = HCP-LCE CDR-L3
GGYDSRTGYVGV

SEQ ID NO: 9 - protein, 132 aa, Gallus sp.
  REGION 1..132: note = NKE14 VH-domain
AVTLDESGGGLQTPGGGLSLVCKGSGFTFSSYNMGWVRQAPGKGLEFVASIDDDGSFTHYGAAVKGRATISRDNGQSTVRLQLNNLRVEDSGIYYCAKSSINGYRCSGGLCVPYITGNIDAWGHGTEVIVSS

SEQ ID NO: 10 - protein, 5 aa, Gallus sp.
  REGION 1..5: note = NKE14 CDR-H1
SYNMG

SEQ ID NO: 11 - protein, 17 aa, Gallus sp.
  REGION 1..17: note = NKE14 CDR-H2
SIDDDGSFTHYGAAVKG

SEQ ID NO: 12 - protein, 23 aa, Gallus sp.
  REGION 1..23: note = NKE14 CDR-H3
SSINGYRCSGGLCVPYITGNIDA

SEQ ID NO: 13 - protein, 454 aa, Gallus sp.
  REGION 1..454: note = HCP-LCE Full-length heavy chain
AVTLDESGGGLQTPGGALSLVCKASGFDFSSYDMAWVRQAPGKGLEWVAGIKSNGPWTNYGAAVKGRATISRDNGQSTVRLQLNNLRAEDTAIYYCAKPGAGYVGWDAAGTIDSWGHGTEVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 212 aa, Gallus sp.
  REGION 1..212: note = HCP-LCE full-length light chain
ALTQPSSVSANLGETVKITCSGGAYSDCGWYQQKSPGSAPVTVIYYNDKRPSDIPSRFSGSTFGSTATLTITGVQAEDEAVYYCGGYDSRTGYVGVFGAGTTLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 15 - protein, 223 aa, Gallus sp.
  REGION 1..223: note = common variably heavy chain
AVTLDESGGGLQTPGGALSLVCKASGFDFSSYDMAWVRQAPGKGLEWVAGIKSNGPWTNYGAAVKGRATISRDNGQSTVRLQLNNLRAEDTAIYYCAKPGAGYVGWDAAGTIDSWGHGTEVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 16 - protein, 212 aa, Gallus sp.
  REGION 1..212: note = Common variable light chain
ALTQPSSVSANPGETVKITCSGGGYDGSYYYGWYQQKSPGSAPVTVIYDNTNRPSNIPSRFSGSLSGSTNTLTITGVQAEDEAVYFCGGYDRSGGIFGGGTTLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 17 - protein, 454 aa, Gallus sp.
  REGION 1..454: note = Knob-into-hole (KiH) variant, heavy chain (knob)
AVTLDESGGGLQTPGGALSLVCKASGFDFSSYDMAWVRQAPGKGLEWVAGIKSNGPWTNYGAAVKGRATISRDNGQSTVRLQLNNLRAEDTAIYYCAKPGAGYVGWDAAGTIDSWGHGTEVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 461 aa, Gallus sp.
  REGION 1..461: note = Knob-into-hole (KiH) variant, heavy chain (hole)
AVTLDESGGGLQTPGGGLSLVCKGSGFTFSSYNMGWVRQAPGKGLEFVASIDDDGSFTHYGAAVKGRATISRDNGQSTVRLQLNNLRVEDSGIYYCAKSSINGYRCSGGLCVPYITGNIDAWGHGTEVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 19 - protein, 212 aa, Gallus sp.; identical to SEQ ID NO: 14
  REGION 1..212: note = Knob-into-hole (KiH) variant, light chain

SEQ ID NO: 20 - protein, 700 aa, Gallus sp.
  REGION 1..700: note = ICI2_outer & NKE14_inner, full length, heavy chain
AVTLDESGGGLQTPGGALSLVCKASGFDFSSYDMAWVRQAPGKGLEWVAGIKSNGPWTNYGAAVKGRATISRDNGQSTVRLQLNNLRAEDTAIYYCAKPGAGYVGWDAAGTIDSWGHGTEVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDGGGGSGGGGSAVTLDESGGGLQTPGGGLSLVCKGSGFTFSSYNMGWVRQAPGKGLEFVASIDDDGSFTHYGAAVKGRATISRDNGQSTVRLQLNNLRVEDSGIYYCAKSSINGYRCSGGLCVPYITGNIDAWGHGTEVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 212 aa, Gallus sp.; identical to SEQ ID NO: 14
  REGION 1..212: note = ICI2_outer & NKE14_inner, full length, light chain

SEQ ID NO: 22 - protein, 699 aa, Gallus sp.
  REGION 1..699: note = NKE14_outer & ICI2_inner, full length, heavy chain
AVTLDESGGGLQTPGGGLSLVCKGSGFTFSSYNMGWVRQAPGKGLEFVASIDDDGSFTHYGAAVKGRATISRDNGQSTVRLQLNNLRVEDSGIYYCAKSSINGYRCSGGLCVPYITGNIDAWGHGTEVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDGGGGSGGGGSAVTLDESGGGLQTPGGALSLVCKASGFDFSSYDMAWVRQAPGKGLEWVAGIKSNGPWTNYGAAVKGRATISRDNGQSTVRLQLNNLRAEDTAIYYCAKPGAGYVGWDAAGTIDSWGHGTEVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 23 - protein, 212 aa, Gallus sp.; identical to SEQ ID NO: 14
  REGION 1..212: note = NKE14_outer & ICI2_inner, full length, light chain

SEQ ID NO: 24 - protein, 212 aa, Gallus sp.; identical to SEQ ID NO: 14
  REGION 1..212: note = One-armed variants (oa), full length, light chain

SEQ ID NO: 25 - protein, 231 aa, Gallus sp.
  REGION 1..231: note = One-armed variants (oa), full length, heavy chain (hole)
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 26 - protein, 700 aa, Gallus sp.
  REGION 1..700: note = One-armed variants (oa), ICI2_outer & NKE14_inner, full length,heavy chain (knob)
AVTLDESGGGLQTPGGALSLVCKASGFDFSSYDMAWVRQAPGKGLEWVAGIKSNGPWTNYGAAVKGRATISRDNGQSTVRLQLNNLRAEDTAIYYCAKPGAGYVGWDAAGTIDSWGHGTEVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDGGGGSGGGGSAVTLDESGGGLQTPGGGLSLVCKGSGFTFSSYNMGWVRQAPGKGLEFVASIDDDGSFTHYGAAVKGRATISRDNGQSTVRLQLNNLRVEDSGIYYCAKSSINGYRCSGGLCVPYITGNIDAWGHGTEVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 700 aa, Gallus sp.
  REGION 1..700: note = One-armed variants (oa), NKE14_outer & ICI2_inner, full length,heavy chain (knob)
AVTLDESGGGLQTPGGGLSLVCKGSGFTFSSYNMGWVRQAPGKGLEFVASIDDDGSFTHYGAAVKGRATISRDNGQSTVRLQLNNLRVEDSGIYYCAKSSINGYRCSGGLCVPYITGNIDAWGHGTEVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDGGGGSGGGGSAVTLDESGGGLQTPGGALSLVCKASGFDFSSYDMAWVRQAPGKGLEWVAGIKSNGPWTNYGAAVKGRATISRDNGQSTVRLQLNNLRAEDTAIYYCAKPGAGYVGWDAAGTIDSWGHGTEVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 690 aa, synthetic construct
AVTLDESGGGLQTPGGTLSLVCRASGFSFSSSDMFWVRQAPGKGLEYVAQISKDGSGKYYGAAVKGRATISRDNGQSTVRLQLNNLRAEDTGIYFCAKSGRGYVTAGRIDTWGHGTEVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDGGGGSGGGGSAVTLDESGGGLQTPGGALSLVCKASGFDFSSYDMAWVRQAPGKGLEWVAGIKSNGPWTNYGAAVKGRATISRDNGQSTVRLQLNNLRAEDTAIYYCAKPGAGYVGWDAAGTIDSWGHGTEVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 212 aa, synthetic construct; identical to SEQ ID NO: 14

SEQ ID NO: 30 - protein, 690 aa, synthetic construct; identical to SEQ ID NO: 28

SEQ ID NO: 31 - protein, 212 aa, synthetic construct; identical to SEQ ID NO: 14

SEQ ID NO: 32 - protein, 122 aa, synthetic construct
AVTLDESGGGLQTPGGTLSLVCRASGFSFSSSDMFWVRQAPGKGLEYVAQISKDGSGKYYGAAVKGRATISRDNGQSTVRLQLNNLRAEDTGIYFCAKSGRGYVTAGRIDTWGHGTEVIVSS

SEQ ID NO: 33 - protein, 5 aa, synthetic construct
SSDMF

SEQ ID NO: 34 - protein, 17 aa, synthetic construct
QISKDGSGKYYGAAVKG

SEQ ID NO: 35 - protein, 13 aa, synthetic construct
SGRGYVTAGRIDT

SEQ ID NO: 36 - protein, 997 aa, synthetic construct
QVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKGGGGSGGGGSGGGGSAVTLDESGGGLQTPGGALSLVCKASGFDFSSYDMAWVRQAPGKGLEWVAGIKSNGPWTNYGAAVKGRATISRDNGQSTVRLQLNNLRAEDTAIYYCAKPGAGYVGWDAAGTIDSWGHGTEVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGSWSHPQFEKGGGSGGGSGGSAWSHPQFEKGGGGSGGGGSGGGGSSEVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSSAGGGGSGGGGSGGGGSSDIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIKR

SEQ ID NO: 37 - protein, 719 aa, synthetic construct
AVTLDESGGGLQTPGGALSLVCKASGFDFSSYDMAWVRQAPGKGLEWVAGIKSNGPWTNYGAAVKGRATISRDNGQSTVRLQLNNLRAEDTAIYYCAKPGAGYVGWDAAGTIDSWGHGTEVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGGSHHHHHHGGGGSGGGGSGGGGSSEVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSSAGGGGSGGGGSGGGGSSDIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIKR

SEQ ID NO: 38 - protein, 740 aa, synthetic construct
AVTLDESGGGLQTPGGALSLVCKASGFDFSSYDMAWVRQAPGKGLEWVAGIKSNGPWTNYGAAVKGRATISRDNGQSTVRLQLNNLRAEDTAIYYCAKPGAGYVGWDAAGTIDSWGHGTEVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGGSWSHPQFEKGGGSGGGSGGSAWSHPQFEKGGGGSGGGGSGGGGSQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIK

SEQ ID NO: 39 - protein, 719 aa, synthetic construct; identical to SEQ ID NO: 37

SEQ ID NO: 40 - protein, 241 aa, synthetic construct
QVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIK

SEQ ID NO: 41 - protein, 242 aa, synthetic construct
SEVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSSAGGGGSGGGGSGGGGSSDIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIKR